(19) United States
(12) Patent Application Publication
(10) Pub. No.: US 2026/0315539 A1
(43) Pub. Date: Oct. 15, 2026

(54) STEM CELLS WITH BRAIN TROPISM

(71) Applicant: UNIVERSITÉ CATHOLIQUE DE LOUVAIN, Louvain-la-Neuve (BE)

(72) Inventors: Pierre SONVEAUX, Sterrebeek (BE); Marine BLACKMAN, Wavre (BE); Marie BEDIN, LYON (FR); Justin RONDEAU, Bruxelles (BE); Tania DE MIRANDA CAPELOA, Schaerbeek (BE); Pascal KIENLEN-CAMPARD, Forest (BE); Justine VAN DE VELDE, Woluwe-Saint-Pierre (BE)

(73) Assignee: UNIVERSITÉ CATHOLIQUE DE LOUVAIN, Louvain-la-Neuve (BE)

(21) Appl. No.: 19/105,453

(22) PCT Filed: Aug. 24, 2023
(86) PCT No.: PCT/EP2023/073225

§ 371 Date: Feb. 21, 2025

(30) Foreign Application Priority Data
Aug. 24, 2022 (EP) 22191920.2

(51) Int. Cl. A61K 35/28; A61K 9/00; A61P 25/00; C07K 14/80; C12N 5/0775

(52) U.S. Cl. CPC A61K 35/28; A61K 9/0019; A61P 25/00; C07K 14/80; C12N 5/0668; C12N 2510/00

(57) ABSTRACT

A population of cells overexpressing the cytochrome c oxidase subunit VIIb (COX7B) protein, for use in treating and/or preventing a brain disease or disorder in a subject in need thereof, and pharmaceutical compositions or kits including the same. Also, a method for inducing the brain tropism of cells, preferably stem cells, by using the overexpression of COX7B.

### FIELD OF INVENTION

The present invention relates to the use of a population of cells overexpressing the cytochrome c oxidase subunit VIIb (COX7B) protein, for use in treating and/or preventing a brain disease.

### BACKGROUND OF INVENTION

Neurodegeneration is a pathological state that results in neural cell death. Although the causes of neurodegeneration may be diverse and not always ascertainable, a large number of brain diseases share neurodegeneration as a common pathological state. For example, Alzheimer's disease, Parkinson's disease, Huntington's disease, and amyotrophic lateral sclerosis (ALS) all cause chronic neurodegeneration, which is characterized by a slow, progressive neural cell death over a period of several years. Acute neurodegeneration is characterized by a sudden onset of neural cell death as a result of ischemia, such as stroke, or trauma, such as traumatic brain injury, or as a result of axonal transection by demyelination or trauma caused, for example, by spinal cord injury or multiple sclerosis. Regardless of the underlying cause, a growing body of evidence indicates that, once neurodegeneration is triggered, the outcome for all these disorders is invariably the same—the ultimate death of neural cells.

Neurodegeneration represents a particularly challenging biological environment for cell therapy. One problem in cell therapeutics is low cell survival (less than 5%) of the cells grafted, as the grafted cells tend to undergo significant cell death shortly after injection in vivo. But most importantly, the main issue is the targeting of the grafted cells: indeed, the brain is relatively isolated from the rest of the organism (notably by the blood-brain barrier, BBB). This implies that the cells must be injected within the brain, which is a particularly heavy and difficult medical intervention: indeed, injecting stem cells in the brain faces two major issues. The first issue is the transplantation procedure itself, which raises safety, ethical and efficacy issues. It is invasive, should ideally be repetitive (i.e., chronic treatment involving several injections at different time points), and spatial administration depends on the capacity to detect brain lesions/degenerative areas. The second issue is the limited capacity of transplanted cells to migrate from the injection site to the widespread area of the affected sites, which is crucial for a therapeutic effect in the central nervous system (see, e.g., De Gioia, R., et al., Neural Stem Cell Transplantation for Neurodegenerative Diseases. Int J Mol Sci, 2020. 21 (9)). Typically, injected cells agglomerate and form clusters at the site of injection in the brain parenchyma, and the grafting efficiency/survival rate of the injected cells is estimated to be less than 5%. Stem cells can also be delivered by lumbar puncture injection into the cerebrospinal fluid. Currently, this procedure is more efficient than intravenous administration, notably because more cells are found in the injured site.

There is thus an unmet need for a solution to target cells to the brain for cell therapy of brain diseases.

The Applicant herein surprisingly provides a solution for increasing the brain tropism of cells.

### SUMMARY

The present invention relates to a population of cells overexpressing the cytochrome c oxidase subunit VIIb (COX7B) protein, for use in treating and/or preventing a brain disease or disorder in a subject in need thereof.

In one embodiment, the cells are stem cells, preferably mesenchymal stem cells or neural stem cells. In one embodiment, the cells are mammalian cells, preferably human cells.

In one embodiment, the COX7B protein is overexpressed at a level so as to induce the brain tropism of said cells. In one embodiment, the cells stably overexpress COX7B protein.

In one embodiment, the COX7B protein is mammalian COX7B protein, preferably human COX7B protein.

In one embodiment, the cells further express at least one surface receptor which binds to a brain-specific ligand.

In one embodiment, the brain disease or disorder is selected from the group comprising or consisting of neurodegenerative diseases, neurological disorders, brain injuries and cerebrovascular diseases. In one embodiment, the brain disease or disorder is selected from the group comprising or consisting of Alzheimer's disease, Parkinson's disease, Huntington's disease, dementia, diffuse Lewy Body dementia, frontotemporal dementia (FTD), Louis body dementia, ataxia, motor neuron disease, amyotrophic lateral sclerosis (ALS), epilepsy and seizures, multiple system atrophy, multiple sclerosis, leukodystrophy, progressive supranuclear palsy, Olivopontocerebellar atrophy (OPCA), Shy-Drager syndrome, Striatonigral degeneration, cortico-basal ganglionic degeneration, Parkinson-ALS-dementia complex of Guam and Pick's disease, amyloidosis, Pick's disease, Lou Gehrig's disease, Creutzfeld-Jakob disease, mild cognitive impairment, syphilis, attention deficit hyperactivity disorder (ADHD), schizophrenia, depression, manic-depression, stress disorder, spinal cord injury, myelitis, Lugeric's disease, traumatic brain injury (TBI), concussion, vascular dementia, hypertension, toxic brain injury such as hypoxia or carbon monoxide poisoning, encephalitis, stroke, brain tumors, brain abscess, autism spectrum disorder and the like.

In one embodiment, the population of cells is to be administered in a therapeutic effective amount to a subject in need thereof. In one embodiment, the therapeutic effective amount is from about 1,000 to about 10,000,000 million of said cells per kg of body weight.

In one embodiment, the population of cells is to be systemically administered to said subject, preferably intravenously injected.

The present invention also relates to a pharmaceutical composition comprising a population of cells overexpressing COX7B protein, and a pharmaceutically acceptable vehicle, for use in the prevention and/or the treatment of a brain disease or disorder.

Another object of the present invention is a combination kit comprising (i) a population of cells overexpressing COX7B protein or a pharmaceutical composition comprising the same and (ii) another therapeutic agent, for use for the prevention and/or the treatment of a brain disease or disorder.

The present invention further relates to a method for inducing the brain tropism of cells, preferably stem cells, comprising a step of overexpressing COX7B protein in said cells.

### Definitions

In the present invention, the following terms have the following meanings:

The term “about”, when preceding a figure, means plus or less 10% of the value of said figure.

The term “identity” refers to a measure of the identity of nucleotide sequences or amino acid sequences. In general, the sequences are aligned so that the highest order match is obtained. “Identity” per se has an art-recognized meaning and can be calculated using published techniques. Methods to determine identity and similarity are codified in computer programs. Computer program methods to determine identity and similarity between two sequences include, but are not limited to, GCG program package, the GAP program. As an illustration, by a polynucleotide having a nucleotide sequence having at least, for example, 95% “identity” to a reference nucleotide sequence is intended that the nucleotide sequence of the polynucleotide is identical to the reference sequence except that the polynucleotide sequence may include an average up to five point-mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

The term “pharmaceutically acceptable excipient” refers to an excipient that does not produce an adverse, allergic or other untoward reaction when administered to an animal, preferably a human. It includes any and all solvents, dispersion media, coatings, antibacterial and antifungal agents, isotonic and absorption delaying agents and the like. A pharmaceutically acceptable carrier or excipient refers to a non-toxic solid, semi-solid or liquid filler, diluent, encapsulating material or formulation auxiliary of any type. For human administration, preparations should meet sterility, pyrogenicity, general safety and purity standards as required by EMA or FDA Office of Biologics standards.

The term “treating” or “treatment” or “alleviation” refers to both therapeutic treatment and prophylactic or preventative measures; wherein the object is to prevent or slow down (lessen) the disease or condition, preferably the brain disease or disorder. Those in need of treatment include those already affected with the disease or condition, preferably the brain disease or disorder, as well as those prone to have the disease or condition, preferably the brain disease or disorder, or those in whom the disease or condition, preferably the brain disease or disorder, is to be prevented. A subject or mammal is successfully “treated” for the disease or condition, preferably the brain disease or disorder, if, after receiving a therapeutic amount of the population of cells for use according to the present invention, the patient shows observable and/or measurable reduction in or absence of one or more of the symptoms associated with the disease or condition, preferably the brain disease or disorder, and/or reduced morbidity and mortality, and/or improvement in quality of life issues. The above parameters for assessing successful treatment and improvement in the disease or condition, preferably the brain disease or disorder, are readily measurable by routine procedures familiar to a physician.

“Therapeutically effective amount” refers to the level or amount of an agent that is aimed at, without causing significant negative or adverse side effects to the target, (1) delaying or preventing the onset of the disease or condition; (2) slowing down or stopping the progression, aggravation, or deterioration of one or more symptoms of the disease or condition; (3) bringing about ameliorations of the symptoms of the disease or condition; (4) reducing the severity or incidence of the disease or condition; or (5) curing the disease or condition. A therapeutically effective amount may be administered prior to the onset of the disease or condition, for a prophylactic or preventive action. Alternatively, or additionally, the therapeutically effective amount may be administered after initiation of the disease or condition, for a therapeutic action.

The term “stem cell” refers to a progenitor cell capable of self-renewal, i.e., can proliferate without differentiation, whereby the progeny of a stem cell or at least part thereof substantially retains the unspecialized or relatively less specialized phenotype, the differentiation potential, and the proliferation competence of the mother stem cell. The term encompasses stem cells capable of substantially unlimited self-renewal, i.e., wherein the capacity of the progeny or part thereof for further proliferation is not substantially reduced compared to the mother cell, as well as stem cells which display limited self-renewal, i.e., wherein the capacity of the progeny or part thereof for further proliferation is demonstrably reduced compared to the mother cell.

The term “subject” or “individual” refers to an animal individual, preferably a mammalian individual, more preferably a human individual. In some embodiments, an individual may be a mammalian individual. Mammalians include, but are not limited to, all primates (human and non-human), cattle (including cows), horses, pigs, sheep, goats, dogs, cats, and any other mammal which is awaiting the receipt of, or is receiving medical care or was/is/will be the object of a medical procedure, or is monitored for the development of a disease or condition, preferably a brain disease or disorder. In some embodiments, an individual may be a “patient”, i.e., a warm-blooded animal, more preferably a human, who/which is awaiting the receipt of, or is receiving medical care or was/is/will be the object of a medical procedure, or is monitored for the development of a disease or condition, preferably a brain disease or disorder. In some embodiments, the individual is an adult (e.g., an individual above the age of 18). In some embodiments, the individual is a child (e.g., an individual below the age of 18). In some embodiments, the individual is a male. In some embodiments, the individual is a female.

### DETAILED DESCRIPTION

The present invention relates to a population of cells overexpressing the cytochrome c oxidase subunit VIIb (COX7B) protein, for use in treating and/or preventing a brain disease or disorder in a subject in need thereof.

In some embodiments, the cells are stem cells.

It is known in the art that stem cells are undifferentiated cells with very high proliferation capabilities, that are able to differentiate into multiple cell types. Stem cells may be totipotent, pluripotent or multipotent. Within the scope of the present invention, the stem cells are preferably multipotent, i.e., the cells can differentiate into multiple cell types in a specific cell lineage.

In a preferred embodiment, the stem cells are mesenchymal stem cells or neural stem cells. In a preferred embodiment, the stem cells are neural stem cells. In certain embodiments, the neural stem cells will give rise to neuronal cells.

As used herein, the terms “neural stem cells” and “neural progenitor cells” are used interchangeably.

In another embodiment, the stem cells are mesenchymal stem cells.

In some embodiments, the stem cells are adult stem cells or embryonic stem cells. In a preferred embodiment, the stem cells are adult stem cells. In certain embodiments, the stem cells are induced pluripotent stem cells (iPSC) or derive from iPSC.

In some embodiments, the cells are mammalian cells, preferably human cells. In some embodiments, the cells are harvested from a mammalian donor, preferably a human donor.

In some embodiments, the stem cells are neurons precursors, oligodendrocyte precursors, astrocyte precursors or microglia precursors. In some embodiments, the stem cells are neuron precursors. In some embodiments, the stem cells are oligodendrocyte precursors. In some embodiments, the stem cells are astrocyte precursors. In some embodiments, the stem cells are microglia precursors.

A skilled person will appreciate that at least some aspects of harvesting cells from donor subjects, in particular human subjects, may be subject to respective legal and ethical norms. By means of example and not limitation, harvesting of cells from a living human donor may need to be compatible with sustenance of further life of the donor.

Accordingly, only a fraction of a cell population may typically be removed from a tissue or organ of a living human donor, e.g., using biopsy or resection, such that an adequate level of physiological functions is maintained in the donor. On the other hand, harvesting of cells from a non-human animal may be considered, but is not obligatorily need compatible with further survival of the non-human animal. For example, the non-human animal may be humanely culled after harvesting of the cells. These and analogous considerations will be apparent to a skilled person and reflect legal and ethical standards.

Cells may be obtained from a donor, preferably a human donor, who has sustained circulation, e.g., a beating heart, and sustained respiratory functions, e.g., breathing lungs or artificial ventilation. Harvesting of cells from such donors is advantageous, since the tissue does not suffer substantial anoxia (lack of oxygenation), which usually results from ischemia (cessation of circulation).

In a preferred embodiment, the cells originate from the subject to be treated. It is to be understood that the purpose of an autologous transplantation (i.e., the administration in an organism of cells from the same organism) is to minimize or abolish the risk of an immune response against the transplant, which would induce the reject of the transplant. The skilled person will appreciate the safety of the transplanted cells.

In another embodiment, the cells originate from a different individual than the subject, preferably and individual of the same species as the subject. In some embodiments, the cells are ReNcell cells.

The population of cells for use according to the invention may be homogenous or heterogenous, preferably homogenous.

In one embodiment, the population of cells for use according to the invention is homogenous. In some embodiments, the population of cells for use according to the invention is substantially free of other cell types. In some embodiments, the population of cells for use according to the invention is substantially free of cells that do not overexpress COX7B. As used herein, “substantially free” means less than 50%, 40%, 30%, 20%, 10%, 9%, 8%, 7%, 6%, 5%, 4%, 3%, 2%, 1%, 0.5%, 0.1%, 0.01%, 0.001% or less.

In another embodiment, the population of cells for use according to the invention is heterogenous. In certain embodiments, the population of cells for use according to the invention comprises at least 2 cell types.

In some embodiments, the population of cells for use according to the invention may be frozen (i.e., cryopreserved) for long term storage. In some embodiments, the population of cells for use according to the invention may be frozen and stored in liquid nitrogen or at any temperature, preferably from about 0° C. to about −196° C., more preferably from about −20° C. to about −196° C., even more preferably from about −80° C.° C. to about −196° C. so long as the cells are able to be used as stem cells after thawing therefrom. In some embodiments, the population of cells for use according to the invention is contacted with at least one cryoprotectant agent, such as, e.g., glycerol, prior to freezing. In one embodiment, the population of cells for use according to the invention may be thawed and expanded further to obtain fresh cells.

In some embodiments, the population of cells for use according to the invention is cultured, i.e., seeded and expanded in vitro. General methods to maintain a cell population in culture are well known in the art. The environment in which the cells are cultured may comprise at least a cell medium, typically a liquid medium, which supports the survival and/or growth of the cells. The term “cell medium” or “cell culture medium” or “medium” refers to an aqueous liquid or gelatinous substance comprising nutrients which can be used for maintenance or growth of cells. Cell culture media can contain serum or be serum-free.

Typically, the medium comprises a basal medium formulation as known in the art. Non-limitative examples of basal media formulations include Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, Medium 199, Waymouth's MB 752/1, StemMacs™ from Miltenyi, Prime-XV from FUJIFILM Irvine Scientific or Williams Medium E, and modifications and/or combinations thereof. Compositions of the above basal media are generally known in the art and it is within the skill of one in the art to modify or modulate concentrations of media and/or media supplements as necessary for the cells cultured. In a preferred embodiment, the medium is a commercially available serum-free medium that supports the growth of stem cells.

Such basal media formulations contain ingredients necessary for mammal cell development, which are known per se. By means of illustration and not limitation, these ingredients may include inorganic salts (in particular salts containing Na, K, Mg, Ca, Cl, P and possibly Cu, Fe, Se and Zn), physiological buffers (e.g., HEPES, bicarbonate), nucleotides, nucleosides and/or nucleic acid bases, ribose, deoxyribose, amino acids, vitamins, antioxidants (e.g., glutathione) and sources of carbon (e.g., glucose, pyruvate, e.g., sodium pyruvate, acetate, e.g., sodium acetate), etc. It will also be apparent that many media are available as low-glucose formulations with or without sodium pyruvate.

For use in culture, basal media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., β-mercaptoethanol. While many basal media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin.

In some embodiments, the cells are maintained in culture conditions so as not to induce differentiation of the cells. In some embodiments, the cells are maintained in culture conditions so as not to induce determination of the cells.

In some embodiments, the COX7B protein is a mammalian COX7B protein, preferably human COX7B protein.

In practice, COX7B, with Entrez Gene ID No. 1349, also refers non-limitatively to Cytochrome C Oxidase Subunit 7B; Cytochrome C Oxidase Subunit 7B, Mitochondrial; Cytochrome C Oxidase Polypeptide VIIb; Cytochrome C Oxidase Subunit VIIb; Cytochrome-C Oxidase Chain VIIb; LSDMCA2 or APLCC.

The cytochrome c oxidase subunit 7B (COX7B) typically has the amino acid sequence as set forth in SEQ ID NO: 1 in humans.

In some embodiments, the COX7B protein has at least 75%, 80%, 85%, 90%, 95%, 96%, 97%, 98%, 99%, 99.5% or 100% sequence identity with SEQ ID NO: 1.

In some embodiments, the COX7B protein is the wild type human COX7B. In some embodiments, the COX7B protein has the amino acid sequence of SEQ ID NO: 1.

In some embodiments, the COX7B protein is a mutant COX7B. In some embodiments, the COX7B protein comprises at least one amino acid mutation compared to the sequence of SEQ ID NO: 1. As used herein, “at least one” means 1, 2, 3, 4, 5, 6, 7, 8, 9, 10, 11, 12, 13, 14, 15, 6, 17, 18, 19 or 20. In some embodiments, “amino acid mutation” comprises substitutions, deletions, insertions, inversions, and combination thereof.

In some embodiments, the mutations on COX7B do not produce any adverse effect on the cell, i.e., the mutations do not alter the viability, proliferation and/or differentiation capacities of the cell. In some embodiments, the mutations are not oncogenic.

The Inventors surprisingly found that COX7B protein was responsible for controlling the brain tropism of cells (see Example).

In some embodiments, the COX7B protein is overexpressed at a level so as to induce the brain tropism of the cells.

As used herein, “induce the brain tropism” means that, when administered to a subject, preferably in the systemic circulation of the subject, an increased proportion of the population of cells according to the present invention homes to the brain of the subject compared to the same type of cells without COX7B overexpression.

In some embodiments, the brain tropism of the population of cells according to the present invention is increased at least 1.5-fold, 2-fold, 3-fold, 4-fold, 5-fold, 6-fold, 7-fold, 8-fold, 9-fold, 10-fold, 100-fold, 1,000-fold, 10,000-fold, 100,000-fold or more compared to the same type of cells without COX7B overexpression. In some embodiments, COX7B overexpression is necessary to induce brain tropism, i.e., the same type of cells without COX7B overexpression have 0% of brain localization.

In some embodiments, at least 5%, 10%, 15%, 20%, 25%, 30%, 35%, 40%, 45%, 50%, 55%, 60%, 65%, 70%, 75%, 80%, 85%, 90%, 95% or 99% of the population of cells according to the present invention localizes to the brain of the subject. In some embodiments, 100% of the population of cells according to the present invention localizes to the brain of the subject.

In some embodiments, the cells overexpress at least 1.2-fold the COX7B protein. It is to be understood that the at least 1.2-fold increase is with respect to COX7B expression levels before overexpression, or with respect to COX7B expression level in an identical population of cells that do not overexpress COX7B. As used herein, “at least 1.2-fold” means 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 10,000-fold, 100,000-fold or more.

Overexpression of COX7B can be controlled and/or measured by methods well known in the art. In some embodiments, COX7B overexpression in the population of cells is measured prior to administration to a subject.

In some embodiments, COX7B overexpression is measured at the protein level. Non-limitative examples of methods that enable quantitation of a specific protein (i.e., COX7B protein) include Western-Blot, immunofluorescence, quantitative flow cytometry, and liquid chromatography-mass spectrometry (LC-MS).

In some embodiments, COX7B overexpression is measured at the RNA level, preferably the messenger RNA (mRNA) level. Non-limitative examples of methods that enable quantitation of a specific RNA (i.e., COX7B RNA, preferably COX7B mRNA) include RNA-seq, quantitative reverse transcription polymerase chain reaction (RT-qPCR), quantitative polymerase chain reaction (or “real-time polymerase chain reaction”, qPCR), and fluorescent nucleic acid probes.

In some embodiments, the cells stably or transiently overexpress COX7B protein. In a preferred embodiment, the cells stably overexpress COX7B protein. In another embodiment, the cells transiently overexpress COX7B protein.

In some embodiments, the COX7B protein is encoded by an exogenous or endogenous nucleic acid. In some embodiments, the exogenous or endogenous nucleic acid comprises the COX7B gene, preferably mammalian COX7B gene, more preferably human COX7B gene. In some embodiments, the COX7B protein is encoded by an exogenous nucleic acid. In some embodiments, the COX7B protein is encoded by an endogenous nucleic acid, typically the COX7B protein is encoded by at least 2 copies of the COX7B gene in the genome of the cell.

In order to achieve overexpression, it is common practice in the art to put the gene that is to be overexpressed under the control of a promoter, preferably a “strong” promoter. In some embodiments, the exogenous or endogenous nucleic acid further comprises a promoter, preferably a strong promoter. Non-limitative examples of strong promoters include cytomegalovirus (CMV) promoter, phosphoglycerokinase (PGK) promoter, and cytomegalovirus/actin/β-globin (CAG) promoter. In order to achieve overexpression, it is also common practice in the art to integrate the gene that is to be overexpressed in a vector containing a selection cassette, such as a selection cassette that confers resistance to a given antibiotic, such as hygromycin.

In some embodiments, COX7B overexpression is achieved by contacting the population of cells with a vector suitable for COX7B overexpression. In some embodiments, the vector comprises at least one nucleic acid as defined herein. In some embodiments, the vector comprises the human COX7B ORF cDNA sequence.

In some embodiments, the vector is an integrating vector or a non-integrating vector.

In some embodiments, the vector is an integrating vector. In some embodiments, the integrating vector is selected from the group comprising or consisting of integrating virus, integrating plasmids, enzymes including engineered transposase or engineered integrase, or genome editing methods including CRISPR-Cas9.

In some embodiments, the integrating vector is an integrating virus selected from the group comprising or consisting of Retroviridae, Adenoviridae, Flaviviridae, Herpesviridae, Hepadnaviridae, Papillomaviridae, Polyomaviridae, Parvoviridae, Arenaviridae, Bornaviridae, Bunyaviridae, Filoviridae and Paramyxoviridae families of viruses, preferably selected from the group comprising or consisting of Retroviridae, Adenoviridae and Flaviviridae families of viruses. In some embodiments, the integrating virus belongs to the family of Retroviridae family. In some embodiments, the integrating virus is a lentivirus.

In some embodiments, a genome editing method is used to insert at least one copy of COX7B gene in the genome of the cells. Non limitative examples of genome editing methods include CRISPR-Cas9, zinc finger nucleases (ZFN), transcription activator-like effector nuclease (TALEN), and the like. Means to implement these methods are well known in the art.

In some embodiments, COX7B overexpression does not affect the function and/or viability of the cell.

In some embodiments, the vector is a non-integrating vector. In some embodiments, the non-integrating vector is viral vector or a non-viral vector.

In some embodiments, the vector is a non-integrating viral vector selected from the group comprising or consisting of adenoviral vector, adeno-associated virus (AAV) vector, integration-deficient lentiviral vector (IDLVs), poxviral vector, herpes simplex virus vector.

In some embodiments, the vector is a non-integrating non-viral vector selected from the group comprising or consisting of plasmid, fosmid, cosmid, artificial chromosome (e.g., human artificial chromosome), nanoparticle (e.g., polymer-based nanoparticle, proteoliposome or lentiviral-like particle), minicircle DNA, and the like. In some embodiments, the non-integrating non-viral vector is a plasmid or a nanoparticle.

In some embodiments, the non-integrating non-viral vector is a plasmid. In some embodiments, the plasmid is a pCMV3 plasmid. In some embodiments, the plasmid is pCMV3 encoding the human COX7B ORF cDNA sequence.

In another embodiment, COX7B overexpression is achieved by contacting the population of cells with a naked nucleic acid expressing COX7B. In some embodiments, the naked nucleic acid enters the cell by a mean selected from the group comprising or consisting of electroporation, sonoporation, ballistic propulsion of DNA-coated particles, and injection.

The person skilled in the art knows techniques for transient or stable expression of a nucleic acid sequence (by, e.g., non-integrating vectors), and other important parameters such as confluence (e.g., transfection at 30-60% confluence) or the duration before a suitable level of expression is reached (7 days, 14 days, or more).

In some embodiments, the cells further express at least one surface receptor which binds to a brain-specific ligand. In some embodiments, the expression of the at least one surface receptor further increases the brain tropism induced by COX7B overexpression.

As used herein, “brain disease or disorder” refers to all kinds of diseases or conditions which occur in the brain of animals, preferably in the brain of humans. As used herein, “degenerative brain disease” or “neurodegenerative disease” is a subset of brain diseases and refers to any brain disease occurring from a degenerative change in nerve cells of the nervous system. The causes of degenerative brain diseases are mostly unknown, and the diseases have slow onset and continuously progress via selective intrusion into associated nerve systems.

In some embodiments, the brain disease or disorder is selected from the group comprising or consisting of neurodegenerative diseases, neurological disorders, brain injuries and cerebrovascular diseases. In some embodiments, the brain disease or disorder comprises neuronal cell death. In some embodiments, the brain disease or disorder comprises or consists of increased neuronal cell death, wherein “increased neuronal cell death” refers to an increase of 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10,000-fold or more compared to a healthy subject.

In some embodiments, the brain disease or disorder is selected from the group comprising or consisting of Alzheimer's disease, Parkinson's disease, Huntington's disease, dementia, diffuse Lewy Body dementia, frontotemporal dementia (FTD), Louis body dementia, ataxia, motor neuron disease, amyotrophic lateral sclerosis (ALS), epilepsy and seizures, multiple system atrophy, multiple sclerosis, leukodystrophy, progressive supranuclear palsy, Olivopontocerebellar atrophy (OPCA), Shy-Drager syndrome, Striatonigral degeneration, cortico-basal ganglionic degeneration, Parkinson-ALS-dementia complex of Guam and Pick's disease, amyloidosis, Pick's disease, Lou Gehrig's disease, Creutzfeld-Jakob disease, mild cognitive impairment, syphilis, attention deficit hyperactivity disorder (ADHD), schizophrenia, depression, manic-depression, stress disorder, spinal cord injury, myelitis, Lugeric's disease, traumatic brain injury (TBI), concussion, vascular dementia, hypertension, toxic brain injury such as hypoxia or carbon monoxide poisoning, encephalitis, stroke, brain tumors, brain abscess, autism spectrum disorder and the like.

In some embodiments, the brain disease or disorder is selected from the group comprising or consisting of Alzheimer's disease, Parkinson's disease, Huntington's disease, dementia, diffuse Lewy Body dementia, frontotemporal dementia (FTD), Louis body dementia, ataxia, motor neuron disease, amyotrophic lateral sclerosis (ALS), epilepsy and seizures, multiple system atrophy, multiple sclerosis, leukodystrophy, progressive supranuclear palsy, Olivopontocerebellar atrophy (OPCA), Shy-Drager syndrome, Striatonigral degeneration, cortico-basal ganglionic degeneration, Parkinson-ALS-dementia complex of Guam and Pick's disease, amyloidosis, Pick's disease, Lou Gehrig's disease, Creutzfeld-Jakob disease, mild cognitive impairment, syphilis, attention deficit hyperactivity disorder (ADHD), schizophrenia, depression, manic-depression, stress disorder and the like.

In some embodiments, the brain disease or disorder is selected from the group comprising or consisting of Alzheimer's disease, Parkinson's disease, Huntington's disease, dementia, diffuse Lewy Body dementia, frontotemporal dementia (FTD), Louis body dementia, ataxia, motor neuron disease, amyotrophic lateral sclerosis (ALS), epilepsy and seizures, multiple system atrophy, multiple sclerosis, leukodystrophy, progressive supranuclear palsy, Olivopontocerebellar atrophy (OPCA), Shy-Drager syndrome, Striatonigral degeneration, cortico-basal ganglionic degeneration, Parkinson-ALS-dementia complex of Guam and Pick's disease, amyloidosis, Pick's disease, Lou Gehrig's disease, Creutzfeld-Jakob disease and the like.

In some embodiments, the brain disease or disorder is selected from the group comprising or consisting of traumatic brain injury (TBI), concussion, vascular dementia, hypertension, toxic brain injury such as hypoxia or carbon monoxide poisoning, encephalitis, stroke, brain tumors, brain abscess, autism spectrum disorder and the like.

In some embodiments, the brain disease or disorder comprises an increase of the permeability of the blood brain barrier (BBB).

In some embodiments, the population of cells compensates the neuronal cell death. In some embodiments, the population of cells compensates at least 5%, 10%, 20%, 30%, 40%, 50%, 60%, 70%, 80%, 90% or 100% of the neuronal cell death induced by the brain disease or disorder.

In some embodiments, administering the population of cells in a subject suffering from a brain disease or disorder increases at least 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 20-fold or more the number of neuronal cells compared to an untreated subject with the same brain disease or disorder.

In some embodiments, the population of cells restores normal brain function in a subject suffering from a brain disease or disorder.

In some embodiments, the population of cells is to be administered in a therapeutic effective amount to a subject in need thereof.

In some embodiments, the therapeutic effective amount is from about 1,000 to about 10,000,000 million of cells per kg of body weight.

In some embodiments, the therapeutic effective amount is from about 2,000 to about 10,000,000 million of cells per kg of body weight, from about 3,000 to about 10,000,000 million of cells per kg of body weight, from about 4,000 to about 10,000,000 million of cells per kg of body weight, from about 5,000 to about 10,000,000 million of cells per kg of body weight, from about 6,000 to about 10,000,000 million of cells per kg of body weight, from about 7,000 to about 10,000,000 million of cells per kg of body weight, from about 8,000 to about 10,000,000 million of cells per kg of body weight, from about 9,000 to about 10,000,000 million of cells per kg of body weight, from about 10,000 to about 10,000,000 million of cells per kg of body weight, from about 20,000 to about 10,000,000 million of cells per kg of body weight, from about 30,000 to about 10,000,000 million of cells per kg of body weight, from about 40,000 to about 10,000,000 million of cells per kg of body weight, from about 50,000 to about 10,000,000 million of cells per kg of body weight, from about 60,000 to about 10,000,000 million of cells per kg of body weight, from about 70,000 to about 10,000,000 million of cells per kg of body weight, from about 80,000 to about 10,000,000 million of cells per kg of body weight, from about 90,000 to about 10,000,000 million of cells per kg of body weight, from about 100,000 to about 10,000,000 million of cells per kg of body weight, from about 200,000 to about 10,000,000 million of cells per kg of body weight, from about 300,000 to about 10,000,000 million of cells per kg of body weight, from about 400,000 to about 10,000,000 million of cells per kg of body weight, from about 500,000 to about 10,000,000 million of cells per kg of body weight, from about 600,000 to about 10,000,000 million of cells per kg of body weight, from about 700,000 to about 10,000,000 million of cells per kg of body weight, from about 800,000 to about 10,000,000 million of cells per kg of body weight, from about 900,000 to about 10,000,000 million of cells per kg of body weight, from about 1,000,000 to about 10,000,000 million of cells per kg of body weight, from about 2,000,000 to about 10,000,000 million of cells per kg of body weight, from about 3,000,000 to about 10,000,000 million of cells per kg of body weight, from about 4,000,000 to about 10,000,000 million of cells per kg of body weight, from about 5,000,000 to about 10,000,000 million of cells per kg of body weight, from about 6,000,000 to about 10,000,000 million of cells per kg of body weight, from about 7,000,000 to about 10,000,000 million of cells per kg of body weight, from about 8,000,000 to about 10,000,000 million of cells per kg of body weight, from about 9,000,000 to about 10,000,000 million of cells per kg of body weight.

In some embodiments, the therapeutic effective amount is from about 1,000 to about 9,000,000 million of cells per kg of body weight, from about 1,000 to about 8,000,000 million of cells per kg of body weight, from about 1,000 to about 7,000,000 million of cells per kg of body weight, from about 1,000 to about 6,000,000 million of cells per kg of body weight, from about 1,000 to about 5,000,000 million of cells per kg of body weight, from about 1,000 to about 4,000,000 million of cells per kg of body weight, from about 1,000 to about 3,000,000 million of cells per kg of body weight, from about 1,000 to about 2,000,000 million of cells per kg of body weight, from about 1,000 to about 1,000,000 million of cells per kg of body weight, from about 1,000 to about 900,000 million of cells per kg of body weight, from about 1,000 to about 800,000 million of cells per kg of body weight, from about 1,000 to about 700,000 million of cells per kg of body weight, from about 1,000 to about 600,000 million of cells per kg of body weight, from about 1,000 to about 500,000 million of cells per kg of body weight, from about 1,000 to about 400,000 million of cells per kg of body weight, from about 1,000 to about 300,000 million of cells per kg of body weight, from about 1,000 to about 200,000 million of cells per kg of body weight, from about 1,000 to about 100,000 million of cells per kg of body weight, from about 1,000 to about 90,000 million of cells per kg of body weight, from about 1,000 to about 80,000 million of cells per kg of body weight, from about 1,000 to about 70,000 million of cells per kg of body weight, from about 1,000 to about 60,000 million of cells per kg of body weight, from about 1,000 to about 50,000 million of cells per kg of body weight, from about 1,000 to about 40,000 million of cells per kg of body weight, from about 1,000 to about 30,000 million of cells per kg of body weight, from about 1,000 to about 20,000 million of cells per kg of body weight, from about 1,000 to about 10,000 million of cells per kg of body weight, from about 1,000 to about 9,000 million of cells per kg of body weight, from about 1,000 to about 8,000 million of cells per kg of body weight, from about 1,000 to about 7,000 million of cells per kg of body weight, from about 1,000 to about 6,000 million of cells per kg of body weight, from about 1,000 to about 5,000 million of cells per kg of body weight, from about 1,000 to about 4,000 million of cells per kg of body weight, from about 1,000 to about 3,000 million of cells per kg of body weight, from about 1,000 to about 2,000 million of cells per kg of body weight.

In some embodiments, the therapeutic effective amount is from about 1,000 to about 10,000,000 cells per kg of body weight.

In some embodiments, the therapeutic effective amount is from about 2,000 to about 10,000,000 cells per kg of body weight, from about 3,000 to about 10,000,000 cells per kg of body weight, from about 4,000 to about 10,000,000 cells per kg of body weight, from about 5,000 to about 10,000,000 cells per kg of body weight, from about 6,000 to about 10,000,000 cells per kg of body weight, from about 7,000 to about 10,000,000 cells per kg of body weight, from about 8,000 to about 10,000,000 cells per kg of body weight, from about 9,000 to about 10,000,000 cells per kg of body weight, from about 10,000 to about 10,000,000 cells per kg of body weight, from about 20,000 to about 10,000,000 cells per kg of body weight, from about 30,000 to about 10,000,000 cells per kg of body weight, from about 40,000 to about 10,000,000 cells per kg of body weight, from about 50,000 to about 10,000,000 cells per kg of body weight, from about 60,000 to about 10,000,000 cells per kg of body weight, from about 70,000 to about 10,000,000 cells per kg of body weight, from about 80,000 to about 10,000,000 cells per kg of body weight, from about 90,000 to about 10,000,000 cells per kg of body weight, from about 100,000 to about 10,000,000 cells per kg of body weight, from about 200,000 to about 10,000,000 cells per kg of body weight, from about 300,000 to about 10,000,000 cells per kg of body weight, from about 400,000 to about 10,000,000 cells per kg of body weight, from about 500,000 to about 10,000,000 cells per kg of body weight, from about 600,000 to about 10,000,000 cells per kg of body weight, from about 700,000 to about 10,000,000 cells per kg of body weight, from about 800,000 to about 10,000,000 cells per kg of body weight, from about 900,000 to about 10,000,000 cells per kg of body weight, from about 1,000,000 to about 10,000,000 cells per kg of body weight, from about 2,000,000 to about 10,000,000 cells per kg of body weight, from about 3,000,000 to about 10,000,000 cells per kg of body weight, from about 4,000,000 to about 10,000,000 cells per kg of body weight, from about 5,000,000 to about 10,000,000 cells per kg of body weight, from about 6,000,000 to about 10,000,000 cells per kg of body weight, from about 7,000,000 to about 10,000,000 cells per kg of body weight, from about 8,000,000 to about 10,000,000 cells per kg of body weight, from about 9,000,000 to about 10,000,000 cells per kg of body weight.

In some embodiments, the therapeutic effective amount is from about 1,000 to about 9,000,000 cells per kg of body weight, from about 1,000 to about 8,000,000 cells per kg of body weight, from about 1,000 to about 7,000,000 cells per kg of body weight, from about 1,000 to about 6,000,000 cells per kg of body weight, from about 1,000 to about 5,000,000 cells per kg of body weight, from about 1,000 to about 4,000,000 cells per kg of body weight, from about 1,000 to about 3,000,000 cells per kg of body weight, from about 1,000 to about 2,000,000 cells per kg of body weight, from about 1,000 to about 1,000,000 cells per kg of body weight, from about 1,000 to about 900,000 cells per kg of body weight, from about 1,000 to about 800,000 cells per kg of body weight, from about 1,000 to about 700,000 cells per kg of body weight, from about 1,000 to about 600,000 cells per kg of body weight, from about 1,000 to about 500,000 cells per kg of body weight, from about 1,000 to about 400,000 cells per kg of body weight, from about 1,000 to about 300,000 cells per kg of body weight, from about 1,000 to about 200,000 cells per kg of body weight, from about 1,000 to about 100,000 cells per kg of body weight, from about 1,000 to about 90,000 cells per kg of body weight, from about 1,000 to about 80,000 cells per kg of body weight, from about 1,000 to about 70,000 cells per kg of body weight, from about 1,000 to about 60,000 cells per kg of body weight, from about 1,000 to about 50,000 cells per kg of body weight, from about 1,000 to about 40,000 cells per kg of body weight, from about 1,000 to about 30,000 cells per kg of body weight, from about 1,000 to about 20,000 cells per kg of body weight, from about 1,000 to about 10,000 cells per kg of body weight, from about 1,000 to about 9,000 cells per kg of body weight, from about 1,000 to about 8,000 cells per kg of body weight, from about 1,000 to about 7,000 cells per kg of body weight, from about 1,000 to about 6,000 cells per kg of body weight, from about 1,000 to about 5,000 cells per kg of body weight, from about 1,000 to about 4,000 cells per kg of body weight, from about 1,000 to about 3,000 cells per kg of body weight, from about 1,000 to about 2,000 cells per kg of body weight.

In some embodiments, the therapeutic effective amount is from about 1,000 to about 100,000,000 cells.

In some embodiments, the therapeutic effective amount is from about 2,000 to about 100,000,000 cells, from about 10,000 to about 100,000,000 cells, from about 100,000 to about 100,000,000 cells, from about 1,000,000 to about 100,000,000 cells, or from about 10,000,000 to about 100,000,000 cells. In some embodiments, the therapeutic effective amount is from about 1,000 to about 10,000,000 cells, from about 1,000 to about 1,000,000 cells, from about 1,000 to about 100,000 cells, or from about 1,000 to about 10,000 cells.

In some embodiments, the population of cells is to be systemically administered to the subject, i.e., injected in the bloodstream of the subject. In a preferred embodiment, the population of cells is to be administered by intravenous injection. In another embodiment, the population of cells is to be administered by intra-arterial or intracardiac injection. In another, less preferred embodiment, the population of cells is to be administered by intramuscular, intradermal or subcutaneous injection.

In some embodiments, the population of cells is maintained in a suitable medium.

In one embodiment, the suitable medium enables the survival of the population of cells and is safe for mammalian administration, preferably for human administration. In some embodiments, the medium provides nutrients to the population of cells. In some embodiments, the medium maintains a suitable osmotic pressure for the population of cells.

In another embodiment, the population of cells is transferred to another medium, herein referred to as injection medium, prior to injection, wherein the other medium enables the survival of the population of cells and is safe for mammalian administration, preferably for human administration, but does not comprise nutrients. In some embodiments, the injection medium is physiological serum. In some embodiments, the injection medium is a biological buffer.

In some embodiments, the medium is sterilized by means known in the art such as, e.g., irradiation. In some embodiments, the sterilization eliminates 99.9%, 99.99%, 99.999% or 100% of microorganisms, preferably 100%. In some embodiments, the medium is sterile. As used herein, “sterile” means that the medium is devoid of bacteria, fungi, archaea and/or protozoa.

In some embodiments, the population of cells is to be administered in combination with another therapeutic agent. Non-limitative examples of therapeutic agents include drugs, pharmaceutical agents, peptides, cells (such as cells different from the cells of the invention) or the likes. In some embodiments, the other therapeutic agent is for treating at least one brain disease. Such therapeutic agents are known by the skilled in the art and are commonly used in medical practice. Non-limitative examples of brain disease treatments include, e.g., acetylcholinesterase inhibitors for the treatment of Alzheimer's disease, anti-inflammatory molecules, antioxidants and the like.

In some embodiments, the other therapeutic agent is administered prior, concomitantly or after the population of cells for use according to the invention. In some embodiments, the other therapeutic agent is administered prior to the population of cells for use according to the invention, preferably between about 1 month and about 1 minute prior to the population of cells for use according to the invention. In some embodiments, the other therapeutic agent is administered concomitantly with the population of cells for use according to the invention. In some embodiments, the other therapeutic agent is administered after the population of cells for use according to the invention, preferably between about 1 minute and about 1 month after the population of cells for use according to the invention.

The present invention further relates to a pharmaceutical composition comprising a population of cells overexpressing COX7B protein, and a pharmaceutically acceptable vehicle, for use in the prevention and/or the treatment of a brain disease or disorder.

In some embodiments, the pharmaceutically acceptable vehicle is selected in a group comprising or consisting of a solvent, a diluent, a carrier, an excipient, a dispersion medium, a coating, and any combinations thereof. The carrier, diluent, solvent or excipient must be “acceptable” in the sense of being compatible with the cell population for use according to the invention, and not be deleterious upon being administered to an individual. Typically, the vehicle does not produce an adverse, allergic or other untoward reaction when administered to an individual, preferably a human individual.

For the particular purpose of human administration, the pharmaceutical compositions should meet general safety and purity standards as required by regulatory offices, such as, for example, the Food and Drugs Administration (FDA) Office or the European Medicines Agency (EMA).

The present invention further relates to a combination kit comprising (i) a population of cells overexpressing COX7B protein or a pharmaceutical composition comprising the same and (ii) another therapeutic agent, for use for the prevention and/or the treatment of a brain disease or disorder.

The present invention also relates to a combination kit comprising (i) means for overexpressing COX7B protein in cells and (ii) another therapeutic agent, for use for the prevention and/or the treatment of a brain disease or disorder. Therefore, the present invention relates to a combination kit comprising (i) a population of cells overexpressing COX7B protein or a pharmaceutical composition comprising the same or means for overexpressing COX7B protein in cells and (ii) another therapeutic agent, for use for the prevention and/or the treatment of a brain disease or disorder.

Means and methods for overexpressing a protein in cells are known from the skilled person in the art. Examples of means for overexpressing COX7B protein in cells include, but are not limited to, vectors comprising at least one coding sequence for COX7B as described hereinabove and reagents for transfection of cells. In some embodiments, the cells where COX7B is overexpressed are the cells of the subject to be treated (autologous cells).

In some embodiments, the cells are stem cells. In a preferred embodiment, the stem cells are mesenchymal stem cells or neural stem cells. In one embodiment, the stem cells are mesenchymal stem cells. In one embodiment, the stem cells are neural stem cells.

In some embodiments, the COX7B protein has at least 75%, 80%, 85%, 90%, 95%, 96%, 97%, 98%, 99%, 99.5% or 100% sequence identity with SEQ ID NO: 1.

In some embodiments, the kit further comprises means to administer the population of cells to a subject in need thereof.

In some embodiments, the kit comprises means to transfect a nucleic acid molecule encoding the COX7B protein in cells, preferably stem cells.

The present invention further relates to a method for treating and/or preventing a brain disease or disorder in a subject in need thereof, comprising administering to the subject a therapeutically effective amount of a population of cells overexpressing COX7B protein or a pharmaceutical composition comprising the same.

In some embodiments, the cells are stem cells. In a preferred embodiment, the stem cells are mesenchymal stem cells or neural stem cells. In one embodiment, the stem cells are mesenchymal stem cells. In one embodiment, the stem cells are neural stem cells. Suitable cells are described hereinabove.

In some embodiments, the method comprises the steps of (i) harvesting a population of stem cells in a subject; (ii) overexpressing COX7B protein in the population of stem cells; and (iii) administering to the subject the population of stem cells overexpressing COX7B protein. Means to overexpress COX7B in a cell are known in the art and are described hereinabove.

In some embodiments, the COX7B protein has at least 75%, 80%, 85%, 90%, 95%, 96%, 97%, 98%, 99%, 99.5% or 100% sequence identity with SEQ ID NO: 1.

In some embodiments, the therapeutic effective amount is from about 1,000 to about 10,000,000 million of cells per kg of body weight.

In some embodiments, the method comprises administering the population of cells to the subject by systemic injection, preferably by intravenous injection. In some embodiments, cells are co-administered with a suitable carrier or solute.

In some embodiments, the method further comprises administering to the subject further therapeutic agent, preferably a therapeutic agent for treating a brain disease or disorder.

Brain diseases and disorders are described hereinabove.

The present invention further relates to a method for inducing the brain tropism of cells, preferably stem cells, comprising a step of overexpressing COX7B protein in the cells. Cells and overexpression of COX7B are described hereinabove.

In some embodiments, the COX7B protein has at least 75%, 80%, 85%, 90%, 95%, 96%, 97%, 98%, 99%, 99.5% or 100% sequence identity with SEQ ID NO: 1.

The present invention further relates to a population of cells overexpressing COX7B protein, for the manufacture of a medicament for the treatment and/or prevention of a brain disease or disorder in a subject in need thereof.

### BRIEF DESCRIPTION OF THE DRAWINGS

FIG. 1A-1F is a combination of schemes, dot plot, histograms and graph showing the validation of the brain tropism of MDA-MB-231-derived brain-seeking variants. FIG. 1A shows a schematic representation of in vivo experiments, where 6-week-old female mice were injected intracardially with 100,000 luciferase- and green fluorescent protein (GFP)-expressing cancer cells on Day 0, imaged once a week to track metastases, and sacrificed at Week 4 for organ collection followed by ex vivo bioluminescence imaging and immunohistochemistry. FIG. 1B shows ex vivo bioluminescence imaging of mouse brains at the end of the protocol illustrated in FIG. 1A. The graph shows brain bioluminescence intensity in mice having received parental MDA-MB-231 cancer cells, 231-BR or 231-BR-2 brain-seeking variants (n=3-9). In FIG. 1C-1E, the migration of MDA-MB-231, 231-BR and 231-BR-2 cells was assayed in transwells towards 1% FBS (n=2-3, FIG. 1C), mouse astrocytes (n=6, FIG. 1D) or human astrocytes (n=6, FIG. 1E). FIG. 1F shows the cell count (%) over time on a SpectraMax i3 spectrophotometer equipped with a MiniMax imaging cytometer (Molecular Devices), after seeding 5,000 cells per well in a 96-wells plate (n=18-19). Data are shown as individual values and medians (FIG. 1B) or as means±SEM (FIG. 1C-1F). * P<0.05, ** P<0.01, *** P<0.005, ns: P>0.05; compared to MDA-MB-231 (FIG. 1B-F); using Mann-Whitney test, one-way ANOVA with Dunnett's post hoc test or two-way ANOVA with Dunnett's post hoc test.

FIG. 2A-2M is a combination of histograms, graph and photograph showing that brain-seeking variants are more oxidative than parental MDA-MB-231 human breast cancer cells. In FIG. 2A-2C, the mitochondrial fuel usage of MDA-MB-231 (FIG. 2A, n=14), 231-BR (FIG. 2B, n=14-16) and 231-BR-2 (FIG. 2C, n=14-15) cancer cells was determined using the Fuel Flex test kit (Agilent) on a Seahorse XF96 bioenergetics analyzer. Data are presented as pie and column graphs, where total fuel usage=100%. In FIG. 2D-2F, glucose consumption (FIG. 2D, n=11-16), lactate production (FIG. 2E, n=16-17) and the lactate/glucose ratio (FIG. 2F, n=13-16) were determined from measurements in deproteinized cell supernatants using an enzymatic CMA600 analyzer. In FIG. 2G-2J, basal (FIG. 2H, n=29-32) and maximal (FIG. 2I, n=28-32) mitochondrial oxygen consumption rates (mtOCRs), as well as the OCR of the cells associated to mitochondrial ATP production (FIG. 2J, n=29-32), were measured using a XF Cell Mito Stress Test Kit (Agilent) on a Seahorse XF96 bioenergetics analyzer. Representative Seahorse traces are shown on FIG. 2G. Transmission electron microscopy pictures of the cells are shown in FIG. 2K (bars=1 μm). FIG. 2L shows mitochondrial density quantified on the graph (n=6-12). FIG. 2M shows mitochondrial DNA/nuclear DNA (mtDNA/nDNA) cell content determined using RT-qPCR (n=5). All data are shown as means±SEM. * P<0.05, *** P<0.005, ns: P>0.05; compared to first column; using one-way ANOVA with Dunnett's post hoc test.

FIG. 3A-3H is a set of histograms showing identification of cyclooxygenase 7b (COX7B) as a candidate protein for the brain tropism of human breast cancer. FIG. 3A shows ALDH9A1 mRNA expression normalized to β-actin (n=5-6) and FIG. 3B shows ALDH9A1 protein expression normalized to vinculin (n=6) in MDA-MB-231, 231-BR and 231-BR-2 cancer cells. FIG. 3C shows FH mRNA expression normalized to β-actin (n=5-6) and FIG. 3D shows FH protein expression normalized to β-actin (n=9). FIG. 3E shows NDUFB8 mRNA expression normalized to β-actin (n=6) and FIG. 3F shows NDUFB8 protein expression normalized to β-actin (n=3). FIG. 3G shows COX7B mRNA expression normalized to β-actin (n=5-6) and FIG. 3H shows COX7B protein expression normalized to β-actin (n=9). All data are shown as means±SEM. * P<0.05, ** P<0.01, *** P<0.005, ns: P>0.05; compared to MDA-MB-231 cells; using one-way ANOVA with Dunnett's post hoc test.

FIGS. 4A-4Q is a combination of histograms and photograph showing the cause-effect relationship between COX7B expression and the selective migration of human breast cancer cells towards astrocytes. In FIG. 4A-4E, migration was assayed in transwells with the 231-BR brain-seeking variant, expressing or not (KO using a CRISP-Cas9 strategy) COX7B, towards 1% FBS (FIG. 4A, n=9), human astrocytes (FIG. 4B, n=3), mouse astrocytes (FIG. 4C, n=5), human hepatocytes (FIG. 4D, n=6) or human bronchial cells (FIG. 4E, n=6). In FIG. 4F-4J, migration was assayed in transwells with the 231-BR-2 brain-seeking variant, expressing or not COX7B, towards 1% FBS (FIG. 4F, n=9), human astrocytes (FIG. 4G, n=5-6), mouse astrocytes (FIG. 4H, n=7), human hepatocytes (FIG. 4I, n=6) or human bronchial cells (FIG. 4J, n=6). In FIG. 4K-4O, migration was assayed in transwells with MDA-MB-231 parental cancer cells, overexpressing or not COX7B, towards 1% FBS (FIG. 4K, n=7), human astrocytes (FIG. 4L, n=9), mouse astrocytes (FIG. 4M, n=6), human hepatocytes (FIG. 4N, n=6) or human bronchial cells (FIG. 4O, n=6). All data are normalized to control (first columns) and are shown as means±SEM. * P<0.05, ** P<0.01, *** P<0.005, ns: P>0.05; using Student's t test. FIG. 4P shows COX7B silencing using CRISPR-Cas9 in 231-BR cells and 231-BR-2 cells. FIG. 4Q shows COX7B overexpression on MDA-MB-231 cells using a pCMV3 vector. Shown are western blots reporting in triplicate on COX7b expression. β-actin served as a loading control.

FIG. 5A-5N is a combination of graphs and histograms showing that COX7B expression drives the oxidative switch of brain-seeking variants. FIG. 5A-5D shows 231-BR brain-seeking variant cells expressing or not (KO using a CRISP-Cas9 strategy) COX7B. Basal (FIG. 5B) and maximal (FIG. 5C) mitochondrial oxygen consumption rates (mtOCRs), as well as the mitochondrial OCR of the cells associated to mitochondrial ATP production (FIG. 5D), were measured using a XF Cell Mito Stress Test Kit (Agilent) on a Seahorse XF96 bioenergetics analyzer (n=20 all). Representative Seahorse traces are shown on FIG. 5A. FIG. 5E-5H shows the same as FIG. 5A-5D but using 231-BR-2 brain-seeking variant cells expressing or not COX7B (n=15-20 all). FIG. 51-5L shows the same as FIG. 5A-5D but using MDA-MD-231 parental cancer cells overexpressing or not COX7B (n=23-32 all). All data are normalized to control (dotted lines) and are shown as means±SEM. ** P<0.01, *** P<0.005, ns: P>0.05; using Student's t test. FIG. 5M-5N show cell counting of 231-BR cells (FIG. 5M) and 231-BR-2 cells (FIG. 5N) with or without COX7B silencing. COX7B silencing does not alter brain-seeking variant cell numbers in vitro. 231-BR and 231-BR-2 cells were either wild-type or silenced for COX7B using a CRISPR-Cas9 strategy. Graphs show cell count (%) over time on a SpectraMax i3 spectrophotometer equipped with a MiniMax imaging cytometer, after seeding 5,000 cells per well in a 96-wells plate (n=3-4). Data are shown as means±SEM. ns: p>0.05 compared to MDA-MB-231 cells; using two-way ANOVA with Dunnett's post hoc test.

FIG. 6A-6L is a combination of dot plots, histograms and graphs showing that COX7B drives the brain tropism of metastatic human breast cancer cells in mice. FIG. 6A-6I shows the brain tropism of 231-BR and 231-BR-2 brain-seeking variants expressing or not COX7B (KO using a CRISP-Cas9 strategy) and of parental MDA-MB-231 human breast cancer cells overexpressing or not COX7B was assessed using the protocol depicted in FIG. 1A. The cells constitutively expressed luciferase and GFP. FIG. 6A-6C shows ex vivo bioluminescence intensity (n=4-10) of mouse brains performed 4 weeks after the intracardiac injection of cancer cells. FIG. 6D-6I shows the number of detected metastases (FIG. 6D, 6F, 6H) and the metastasis-positive surface area per mouse (FIG. 6E, 6G, 6I) (n=4-10). FIG. 6J-6L shows overall survival of patients with breast cancer (FIG. 6J, all types combined, 1090 patients), lung cancer (FIG. 6K, all types combined, 1925 patients) and renal clear cell carcinoma (RCC, FIG. 6L, 530 patients). Data are shown as individual values and medians (FIG. 6A-6C), means±SEM (FIG. 6D-6I) or individual values (FIG. 6J-6L). * P<0.05, ** P<0.01, ns: P>0.05; using Mann-Whitney test (FIG. 6A-6C), Student's t test (FIG. 6D-6I) or log rank test (FIG. 6J-6L).

FIG. 7A-7C is a combination of histograms showing that COX7B silencing does not alter the metabolic plasticity of brain-seeking variant cells. 231-BR and 231-BR-2 cells were either wild-type or silenced for COX7B using a CRISPR-Cas9 strategy. (FIG. 7A-7C) Mitochondrial fuel usage of parental MDA-MB-231 cells, 231-BR cells expressing or not COX7B and 231-BR-2 expressing or not COX7B. Column graphs show mitochondrial glucose (FIG. 7A; n=7-15), glutamine (FIG. 7B; n=12-16) and fatty acid (FIG. 7C; n=14-16) usage, where total fuel (glucose+glutamine+fatty acids) usage=100% for each cell line. Data are shown as means±SEM. * P<0.05, ** P<0.01, *** P<0.005, ns: P>0.05 compared to MDA-MB-231 cells; using one-way ANOVA with Dunnett's post hoc test.

FIG. 8 is a photograph showing that ReNcell VM human neural progenitor cells can be engineered to overexpress human COX7B. ReNcell VM human neural progenitor cells were transfected or not with a pCMV3 plasmid encoding the human COX7B cDNA ORF. Parental cells are Rencell VM and the cells overexpressing COX7B were called ReNcell VM COX7B OE cells. Cells were allowed to recover 24 h after transfection in proliferation medium. Cells were then selected with 10 μg/mL hygromycin for 14 days, generating ReNcell VM COX7B OE clones that were individually tested by western blotting for the expression of the COX7B protein (detected at 9 kD using western blotting). β-actin expression (detected at 42 kD) served as a loading control. The clones overexpressing COX7B (10, 11, 12, 13, 14, 15, 16, 17, 18, 19, 21, 22, 23 and 24) compared to the parental cells were pooled together to create the ReNcell COX7B OE cell line.

FIG. 9A-9H is a combination of histograms and photographs showing that human stem cells overexpressing COX7B have a better homing to damaged brains after injection in the blood stream than the parental stem cells that do not express COX7B. 100,000 ReNcell VM human neural stem cells constitutively expressing GFP, 100,000 ReNcell VM COX7B OE human neural stem cells constitutively expressing GFP or no cells (control) were injected in the blood stream of nude mice previously treated with LPS to induce brain damage. Mice were sacrificed 3 days later by cervical dislocation under terminal anesthesia, and their brains were harvested, cut along the separation between right and left hemispheres, and embedded in paraffin. (FIG. 9A-9F) Shown are representative images of brain sections immunostained for GFP. For visualization, each positive cell is represented by a black dot. (FIG. 9A) Representative image of the 2 brain hemispheres of a control mouse (scale bar=1 mm). (FIG. 9B) Representative image of the brain cortex of a control mouse at higher magnification (scale bar=0.2 mm). (FIG. 9C) Representative image of the 2 brain hemispheres of a mouse injected with ReNcell VM cells (scale bar=1 mm). (FIG. 9D) Representative image of the brain cortex of a mouse injected with ReNcell VM cells at higher magnification (scale bar=0.2 mm). (FIG. 9E) Representative image of the 2 brain hemispheres of a mouse injected with ReNcell VM COX7B OE cells (scale bar=1 mm). (FIG. 9F) Representative images of the brain cortex of a mouse ReNcell VM COX7B OE at higher magnification (scale bar=0.2 mm). (FIG. 9G) Graph showing the number of GFP-positive cells per brain hemisphere in control mice, mice injected with ReNcell VM cells in the blood stream, and mice injected with ReNcell VM COX7B OE cells in the blood stream. (FIG. 9H) Graph showing the number of GFP-positive cells per whole brain in control mice, mice injected with ReNcell VM cells in the blood stream, and mice injected with ReNcell VM COX7B OE cells in the blood stream.

### EXAMPLES

The present invention is further illustrated by the following examples:

### Example 1

### Materials and Methods

### Cells and Cell Culture

Parental MDA-MB-231 human triple-negative breast adenocarcinoma cancer cells were from Caliper (catalogue #119369). MDA-MB-231-derived brain-seeking variants 231-BR and 231-BR-2 were from the National Cancer Institute, Bethesda and Indiana University School of Medicine, Indianapolis, respectively. Cells were routinely cultured in DMEM containing glutaMAX and 4.5 g/L glucose (ThermoFisher; catalogue #61965026) supplemented with 10% FBS, and maintained in a 37° C. in a 5% CO₂ humidified atmosphere. Cells were authenticated using short tandem repeat (STR) profiling (Eurofins Genomics).

Human astrocytes (T0281, expressing hTERT), mouse astrocytes (T0289, expressing the SV40 large T antigen), human hepatocytes (T0063, expressing HPV E6/E7, hTERT and MycT58A) and human bronchial epithelial cells (T0753, expressing hTERT and Cdk4) used in migration assays were immortalized cells from Applied Biological Materials Inc. (ABM). Astrocytes were routinely cultured in DMEM with glutaMAX and 4.5 g/L glucose (ThermoFisher; catalogue #61965026) supplemented with 10% FBS and 5% astrocyte growth supplement (Sanbio; catalogue #1852; for human astrocytes only). Hepatocytes were maintained in PriGrow IX medium (ABM; catalogue #TM019) supplemented with 10% FBS, then progressively transferred to the same medium as cancer cells. Bronchial cells were maintained in Prigrow X medium (ABM; catalogue #TM0753), then progressively transferred to bronchial epithelial cell growth medium (BEGM; Lonza; catalogue #CC-3170).

### Genetic Manipulations

For constitutive luciferase and GFP expression, cells were infected with lentiviruses carrying the luciferase and GFP sequences along with puromycin resistance gene (Amsbio; catalogue #LPV020). Briefly, 70-80% confluent cells in a 24-wells plate were transduced with 2 μL per well of the lentivirus solution in 1 mL medium with 10 μL/mL polybrene. Cells were selected by a 48-72 h incubation with 1 μg/mL puromycin (InvivoGen), and FACS-sorted for GFP expression on a Becton Dickinson FACSAriaIII system.

COX7B gene silencing was performed using a CRISPR-Cas9 strategy following Zhang's laboratory protocol with pSpCas9 (BB)-2A-Puro (pX459; Addgene; catalogue #62988, puromycin selection) or pU6-(BbsI)_CBh-Cas9-T2A-mCherry (Addgene; catalogue #64324, red fluorescence selec-tion) plasmids. These plasmids contain both Cas9 and gRNA expression cassettes, with BbsI restriction sites for the insertion of gRNA sequences. Prevalidated guide RNA (gRNA) sequences were chosen in the GenScript genome-wide database for a non-overlapping duo of gRNAs: 5′-AGCGCACTAAATCGTCTCCA-3′ (SEQ ID NO: 15) and 5′-GAGTTACCCCAAAGGAATGG-3′ (SEQ ID NO: 16). Sticky ends were created for insertion in the vector plasmids, with CACCG at the 5′ of the gRNA sense sequence, and AAAC at the 5′ and C at the 3′ of the gRNA antisense sequence. gRNA oligonucleotides (Eurogentec) were annealed into double-stranded DNA with 1 μL of stock solutions containing 100 μM of each sense and anti-sense oligonucleotides in 2 μL of 5× T4 ligase buffer (ThermoFisher; catalogue #46300018), 0.5 μL of T4 PNK (BIOKE; catalogue #M0201) and 5.5 μL of DNase/RNase-free distilled water. Incubation times were 37° C. for 30 min, followed by 95° C. for 5 min, and then de-creasing 5° C./min until reaching 25° C. The Golden Gate DNA Assembly protocol was then used for inserting 1 μL of annealed gRNA at 1 μM into 100 ng of vector plasmid, in a solution containing 5 μL of 10× Fast Digest buffer (ThermoFisher; catalogue #B64), 0.5 μL of ATP 0.1 M (ThermoFisher; catalogue #R1441), 0.5 μL of BSA 10 mg/mL (Promega; catalogue #R396D), 1 μL of restriction enzyme BpiI (ThermoFisher; catalogue #FD1014) and 2 μL of T4 ligase 5 U/μL (ThermoFisher; catalogue #EL0014) in a total volume of 50 μL completed with water. The mixture was incubated for 20 cycles at 37° C. for 5 min and 20° C. for 5 min, followed by 80° C. for 20 min. Five microliters of the resulting solution were used for TOP10 bacteria (ThermoFisher; catalogue #C404003) transformation using prewarmed LB agar plates (ThermoFisher; catalogue #22700-025) containing 100 μg/mL of ampicillin following manufacturer's instructions. Single colonies were inoculated in LB broth (ThermoFisher; catalogue #12780-052) with 50 μg/mL ampicillin, and incubated at 37° C. overnight. Plasmid DNA was then collected with the Pure Yield Plasmid Miniprep System (Promega; catalogue #A1223), and its concentration obtained using a NanoDrop device (ThermoFisher). Sequences were verified by Sanger Sequencing (Genewiz, Leipzig, Germany). Cells at 70-80% confluence were transfected with the Lipofectamine LTX/Plus transfection kit (ThermoFisher; catalogue #15338100) or with the jetOPTIMUS kit (Westburg; catalogue #117-01). The first kit was used with 0.25 μg of each gRNA plasmid, for a total of 0.5 μg of DNA in 100 μL of OptiMEM, containing 0.5 μL of Plus and 2.25 μL of lipofectamine LTX, with incubation times of 15 and 30 min, respec-tively. The second kit was used with 0.5 μg of each gRNA plasmid, for a total of 1 μg of DNA, with 1 μL of reagent in 200 μL of buffer for each 6-wells plate well. Cells were se-lected with puromycin (1 μg/mL)-containing medium for 48-72 h or FACS-sorted for mCherry fluorescence 48 h after transfection on a Becton Dickinson FACSAriaIII system.

The human untagged COX7B cDNA ORF Clone in expression vector pCMV3 (Bio-Connect; catalogue #HG20762-UT) was used to overexpress COX7b in MDA-MB-231 cancer cells. Cells at 70-80% confluence were transfected with 10 μg of the plasmid and Lipofectamine 3000 (ThermoFisher; catalogue #L3000001) in a 10 cm-dish, allowed to recover the next day in fresh DMEM containing glutaMAX and 4.5 g/L glucose (ThermoFisher; catalogue #61965026) supplemented with 10% FBS, and selected with hygromycin (400 μg/ml)-containing medium for 10 days. Medium was renewed every 3-4 days. Colonies individually picked, expanded, and tested by western blotting for the expression of COX7b.

### Metastatic Take in Mice

On day 0, 6-weeks-old female NMRI nude mice (Janvier) received image-guided intraventricular injections of 100,000 cells, using a Vevo 2100 imaging system (FUJIFILM VisualSonics) equipped with a 30 MHz transducer. Briefly, mice were anesthetized (80 mg/Kg ketamine and 8 mg/Kg xylazine), secured on the animal platform in supine position, and their thorax was shaved. Two-dimensional (2D) parasternal long-axis ultrasound images of the left ventricle were acquired, in order to ascertain the optimal point in the apex of the heart for the intraventricular injection. A microinjector system with a 26G hypodermic needle was used to perform a precise echocardiography-guided intraventricular injection of 100,000 cancer cells constitutively expressing luciferase and GFP. Following injection, the blood flow within the left ventricle was closely observed (and images were recorded), confirming that the cells were successfully injected intraventricularly. All mice were followed-up for a few minutes with echography to ascertain that the injection did not lead to any injury and were closely monitored until recovery. Metastasis development was monitored using a Xenogen IVIS 50 bioluminescence imaging system (PerkinElmer), and quantified with the Living Image software (PerkinElmer). Every week, mice were injected i.p with 0.15 mg/g bodyweight of luciferin (PerkinElmer) and anesthetized using isoflurane after a 10-min incubation time. Chemiluminescence was detected with 1-12 seconds acquisition time. Mice were sacrificed after 4 weeks by cervical dislocation under terminal anesthesia, and organ chemiluminescence was acquired ex vivo before fixation in 4% paraformaldehyde (PFA).

### Cell Migration and Invasion

Transwell inserts (Corning; catalogue #353097) were used to measure cell migration and invasion capacities. FBS 1% (general migration/invasion) or cells (astrocytes, hepatocytes, bronchial cells) seeded in the lower chamber were used as chemoattractants. Invasion was assessed by coating the inserts with 250 μg/mL of Matrigel (Corning; catalogue #356231) for 2 h at 37° C., and migration without Matrigel coating. Fifty thousand cells were seeded in the upper chamber of each transwell in 500 μL of serum-free culture medium, while the lower chamber contained FBS 1% (general migration/invasion) or confluent nonmalignant astrocytes, hepatocytes, or bronchial cells that were used as chemoattractants. Tested cells were allowed to migrate/invade for 24 h in a 37° C. and 5% CO₂ humidified atmosphere. At the end of the assay, cells were fixed with 4% PFA for 10 min and PBS, rinsed 3 times with PBS, and immobile cells (upper compartment of the insert) were wiped away. Mobile cells were stained with DAPI for 30 min, rinsed 3 times with PBS and imaged at 5× on an AxioVert microscope equipped with an AxioCam-MRc camera (Zeiss). Two images were taken per well, covering the upper and lower halves each. Nuclei were counted using QuPath software version 0.1.2. with the Positive Cell Detection analysis tool.

### Cell Numbers

Cell numbers were determined over time on a SpectraMax i3 spectrophotometer equipped with a MiniMax imaging cytometer (Molecular Devices), after seeding 5,000 cells per well in a 96-wells plate. Results were normalized to initial cell numbers.

### Metabolic Assays

Oxygen consumption rates (OCRs) were measured on a Seahorse XF96 bioenergetics analyzer using the XF Cell Mito Stress Test Kit and the Fuel Flex test kit following manufacturer's instructions (Agilent Technologies). Glucose and lactate concentrations were measured using an enzymatic CMA600 analyzer (Aurora Borealis), according to manufacturer's instructions, in the supernatant of 150,000 (for 24 h assays) and 250,000 (for 48 h assays) cells were seeded in exactly 1 mL of culture medium. Wells containing medium only were used as controls for the calculation of glucose consumption and lactate production. The ATP content of 10.000 cells per well (96-wells plate) was measured using the Cell titer Glo assay of Promega (catalogue #G7570). All metabolic measurements were normalized to total protein content determined after overnight incubation with 0.5 M NaOH using the Bio-Rad protein assay (catalogue #5000006) on a SpectraMax i3 spectrophotometer equipped with a MiniMax imaging cytometer.

### Electron Microscopy

Cells were collected and resuspended in 400 μL of a 2.5% glutaraldehyde solution containing 0.1 M of sodium cacodylate at pH 7.4 in a pyramidal BEEM capsule (Agar Scientific, catalogue #G360). Samples were then processed as previously disclosed. Images were acquired on a TECNAI G² 20 LaB6 transmission microscope (Field Electron and Ion Company).

### Mitochondrial Abundance and Mitochondrial DNA Content

Mitochondrial DNA (mtDNA) content was measured using RT-qPCR as previously described. Total DNA was isolated with a QIAmp DNA kit (Qiagen, Antwerp, Belgium). The 12S-rRNAA mitochondrial gene (forward primer: 5′-GTA CCC ACG TAA AGA CGT TAG G-3′ (SEQ ID NO: 12); reverse primer: 3′-TAC TGC TAA ATC CAC CTT CG-5′ (SEQ ID NO: 13); labeled probe: 5′-CCC ATG AGG TGG CAA GAA AT-3′ FAM (SEQ ID NO: 14) in parallel with nuclear gene RNAseP (RNAseP VIC-labeled probe; ThermoFisher Scientific; catalogue #4401631) were then analyzed by RT-qPCR (50 ng of sample and 1 μL of each primer pair [10 μM]), with TaqMan universal master mix II with UNG (ThermoFisher). For presentation, mtDNA content was normalized to nuclear DNA (nDNA) content.

### Microarray Database Analysis

According to Gene Expression Omnibus (GEO) and reference, database #GSE66495 reports on the whole genome expression, determined using Illumina HumanHT-12 V4 expression beadchips, of MDA-MB-231 parental cells and tissue-specific metastatic variants derived thereof (including 231-BR) that were maintained in MEM with 10% FBS. For database reanalysis, metabolic genes related to glycolysis, OXPHOS and the TCA cycle were first extracted. Next, only genes that were differentially expressed (p<0.05, using one-way ANOVA) in brain (231-BR), adrenal (ADMD-231), bone (BMD-231) and/or lung (LMD-231) metastatic variants compared to parental MDA-MB-231 cells were retained. They are displayed in Table 1. The genes that were differently expressed (p<0.05, using one-way ANOVA) in 231-BR versus ADMD-231, BMD-231 and LMD-231 cells were then identified. Expression changes were independently confirmed by RT-qPCR using fresh lysates from brain-seeking variants 231-BR and 231-BR-2, and MDA-MB-231 parental cells.

### RT-qPCR

Total RNA was collected using the NucleoSpin RNA kit (Filter Service; catalogue #740955.50), quantified with a NanoDrop 1000 Spectrophotometer (Thermo Fisher), and reverse transcribed in cDNA with the RevertAid First Strand cDNA synthesis kit (Thermo Fisher; catalogue K1621) using the same quantity (500-1,000 ng) for all RNA samples and a 90-min incubation time. cDNAs were diluted 1:10 in DNase/RNase-free distilled water (Thermo Fisher), and 2 μL were used with 5 μL of 2× Takyon qPCR Master Mix, 0.2 μL of each primer (10 μM) and completed to 10 μL with water for RT-qPCR analysis (ViiA 7417 Real-Time instrument, ThermoFisher). Primers were: ALDH9A1 Forward 5′-AAG GAG CAG GGT GCT AAA GT-3′ (SEQ ID NO: 2) and Reverse 5′-TCG TCT CTG CAA TTA GTT AAT ACA C-3′ (SEQ ID NO: 3); FH Forward 5′-TGC CAA CCC CAG TTA TTA AAG C-3′ (SEQ ID NO: 4) and Reverse 5′-CTT CAG CTA CCT CAT CTG CTG-3′ (SEQ ID NO: 5); NDUFB8 Forward 5′-CGG ATG ATG GCA TGG GGT A-3′ (SEQ ID NO: 6) and Reverse 5′-GGT GCC AGT GCA TCG GTT-3′ (SEQ ID NO: 7); and COX7B Forward 5′-TAC CTG AAG CGA ATT GGC AC-3′ (SEQ ID NO: 8) and Reverse 5′-GCT TCG AAC TTG GAG ACG AT-3′ (SEQ ID NO: 9), and β-actin Forward 5′-CCC GCG AGC ACA GAG C-3′ (SEQ ID NO: 10) and Reverse 5′-TCA TCA TCC ATG GTG AGC TGG-3′ (SEQ ID NO: 11). All gene expression data were normalized to β-actin gene expression.

### Western Blotting

Western blotting was performed after protein collection in RIPA buffer containing phosphatase (PhosSTOP) and protease (proteases inhibitor cocktail) inhibitors. Membranes were incubated overnight with primary rabbit antibodies against ALDH9A1 (Proteintech; catalogue #26621), FH (BIOKE; catalogue #4567S), NDUFB8 (Proteintech; catalogue #14794), COX7B (Abcam; catalogue #ab137094), Vinculin (BIOKE; catalogue #4650S); or mouse antibody against β-actin (Sigma-Aldrich; #A5441). Staining was revealed with an Amersham Imager 600 (GE Healthcare). All data are normalized to vinculin or β-actin expression.

### Immunohistochemistry

Brains were collected, cut along the separation between right and left hemispheres, and embedded in paraffin. Sections (5 μm-thick) were performed from the center of each hemisphere to produce 10 slides for each sample. For each hemisphere, 3 slides from the beginning, the middle and the end were used for immunostaining, and the process was repeated up to 3 times in order to analyze slices representative of the whole brain.

Brain sections were immunostained for GFP (Bio-Techne; catalogue #600-308; BIOKE; catalogue #2956), with a secondary Envision anti-rabbit antibody coupled to HRP (Agilent; catalogue #K4003) and hematoxylin and eosin counterstaining. Slides were scanned at 20× magnification with a SCN400 bright field Slide Scanner (Leica Biosystems). Metastasis number and surface area were determined using cytomine (cytomine.org) and the QuPath software version 0.1.2.

### Clinical Database Analysis

Overall Survival (OS) curves were generated on Kaplan-Meier plotter (kmplot.com) with the auto select best cutoff for the 202110 Affy ID (COX7B) on the RNA-seq mRNA dataset (breast cancer and renal clear cell carcinoma in pan-cancer) and on gene chip mRNA data sets for lung cancers. Sources for the databases include GEO, EGA and TCGA.

### Statistics

Data are shown as means±SEM (error bars are sometimes smaller than symbols) or as individual values with the median. ‘n’ indicates the total number of replicates per group/condition. Graphpad Prism version 9.2.0. was used for statistical analyses. Mann-Whitney, Student's t-test, one-way ANOVA and two-ways ANOVA were used where indicated. P<0.05 was considered to be statistically significant.

### Results

Validation of Brain-Seeking Variant Models Derived from Human MDA-MB-231 Triple-Negative Breast Cancer Cells

The objective of the set of experiments presented herein was to identify metabolic protein(s) responsible for the brain tropism of human metastatic breast cancer. As models, MDA-MB-231 triple-negative breast cancer (TNBC) cells were used, as well as two independently derived brain-seeking variant cell lines 231-BR and 231-BR-2 that were generated by serial cycles of in vivo selection in mice. The selection protocol involved intracardiac cancer cell injection in the left ventricle of female nude mice, surgical isolation, expansion of metastatic cancer cells retrieved from the brain, and intracardiac injection of these cells in next animals for several rounds until metastatic dissemination became restricted to the brain.

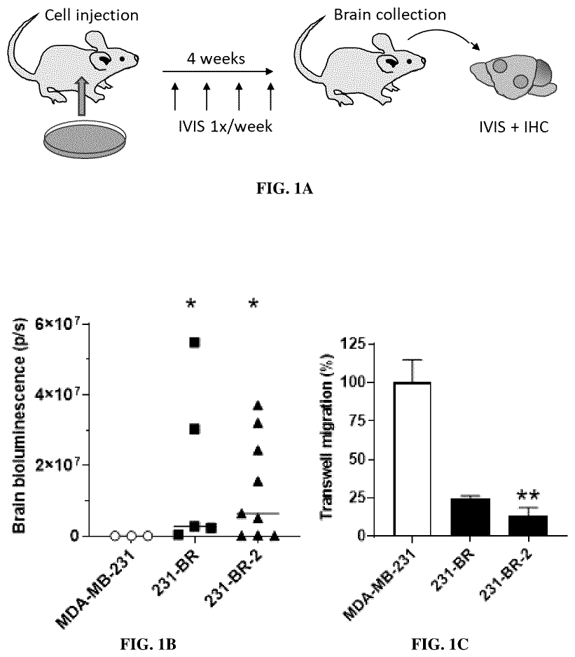

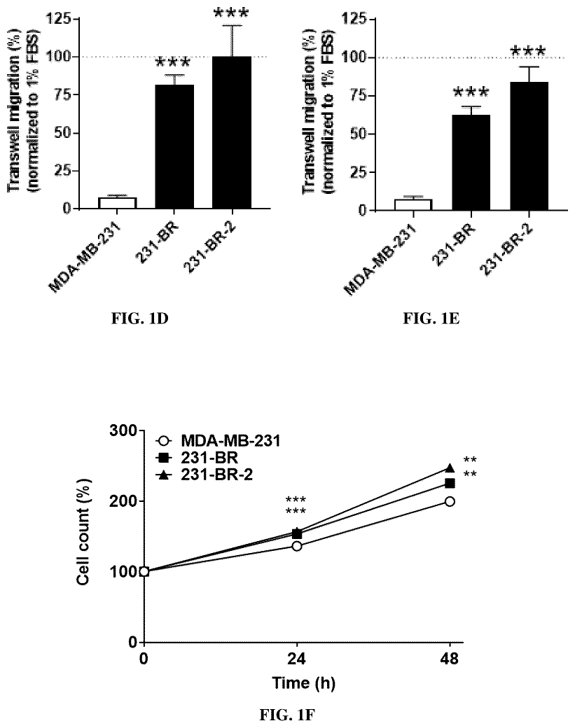

To identify metabolic drivers of brain-specific metastasis, the validity of the two model cell lines was first ascertained in vitro and in vivo. Short tandem repeat (STR) profiling confirmed that all variants were genomically similar to parental MDA-MB-231 cells (data not shown). For in vivo assays, cells were infected with lentiviruses to constitutively express luciferase and green fluorescent protein (GFP). An intracardiac injection of 100,000 MDA-MB-231 parental cells (FIG. 1A) did not generate brain metastases in female nude mice, whereas the use of either 231-BR or 231-BR-2 variant yielded metastases in most animals (4/5 for 231-BR and 6/9 for 231-BR-2) 4 weeks after injection, which were detected by ex vivo bioluminescence imaging on isolated brains (FIG. 1B).

For in vitro model validation, a transwell assay was developed, and aimed to test the general and organotropic migration of the human breast cancer cells in the upper well towards 1% FBS or towards living immortalized nonmalignant astrocytes in the lower well, respectively. Because the selection of the brain-seeking variants was made in mice, both mouse and human astrocytes were tested as attractants. While parental MDA-MB-231 cells had a higher capacity to migrate towards 1% FBS compared to the two brain seeking variants, conversely, 231-BR and 231-BR-2 cells migrated much more efficient towards mouse or human astrocytes (FIG. 1C-1E), thus validating their preferential tropism for the brain. Of note, both brain-seeking variants were slightly yet significantly more proliferative than parental cells, as determined by direct cell counting over time (FIG. 1F).

### Brain-Seeking Variants of MDA-MB-231 Undergo an Oxidative Switch

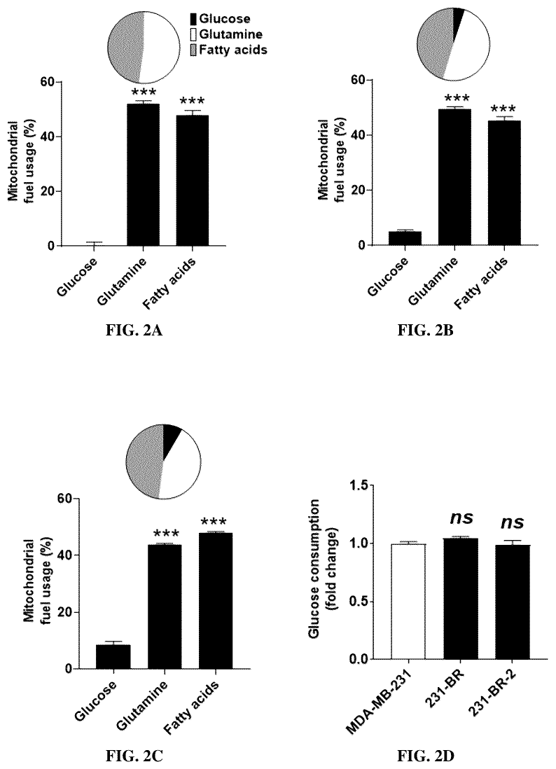

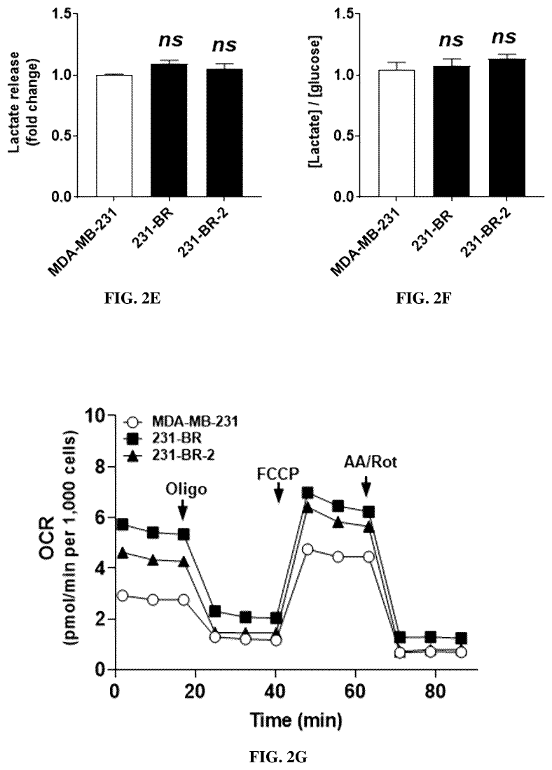

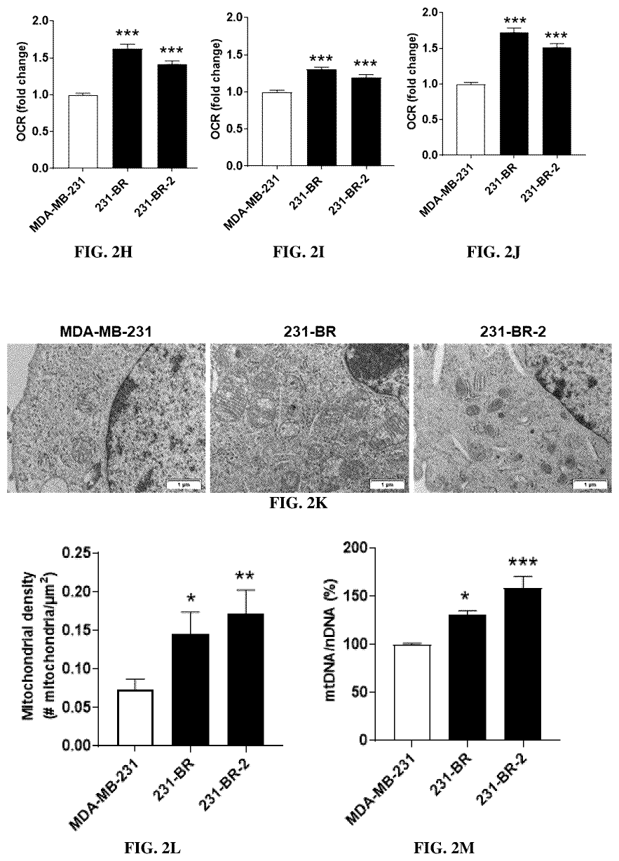

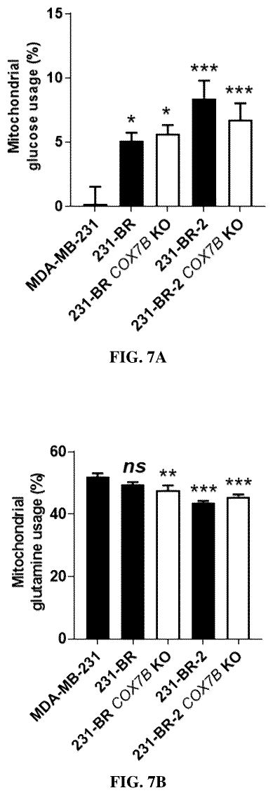

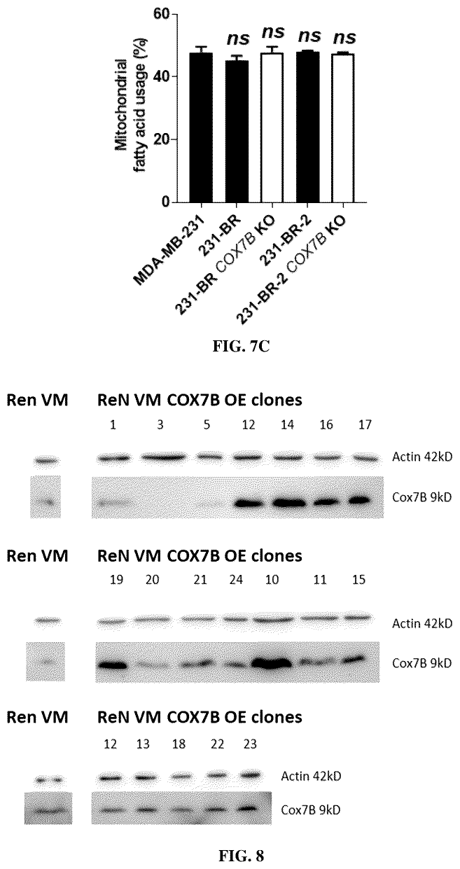

Using Seahorse oximetry and the dedicated Fuel Flex test kit of Agilent, the oxidative metabolic preferences of parental and brain-seeking variant cells determined. The assay involves sequential inhibition of glucose-fueled (using 2 μM mitochondrial pyruvate carrier inhibitor UK5099), glutamine-fueled (using 3 μM glutaminase 1 inhibitor BPTES) and lipid-fueled (using 4 μM carnitine palmitoyl-transferase 1A inhibitor Etomoxir) OXPHOS. Results (FIG. 2A-2M and FIG. 7A-7C) showed that OXPHOS in MDA-MB-231 cells was supported almost equally by glutamine (52%) and fatty acids (48%), but not at all by glucose, whereas OPXHOS in the two brain-seeking variants was supported not only by glutamine and fatty acids but also by glucose (5.1±0.6% for 231-BR and 8.4±1.4% for 231-BR-2 cells) (FIG. 2A-2C). Glucose uptake, lactate release and, hence, the glycolytic ratio ([glucose]/[lactate]) were unchanged in full medium (DMEM containing glutaMAX, 4.5 g/L glucose and 10% FBS) (FIG. 2D-2F). Therefore, changes in the capacity to use OXPHOS fuels reflected an increased oxidative flexibility of brain-seeking variants compared to parental cells, rather than an increased dependency on glucose. Accordingly, both 231-BR and 231-BR-2 cell lines presented improved basal and maximal respiration activities as well as an improved oxidative ATP production compared to parental cells (FIG. 2G-J).

The oxidative switch evidenced in brain-seeking variants was linked to qualitative and quantitative changes affecting mitochondria. Qualitatively, electron microscopy revealed enlarged mitochondria in 231-BR compared to parental cells, whereas they were smaller but more abundant in 231-BR-2 compared to MDA-MB-231 cells (FIG. 2K-2M). The mitochondrial to nuclear DNA ratio (mtDNA/nDNA) was determined using RT-qPCR, revealing a significantly increased mtDNA abundance in both 231-BR and 231-BR-2 compared to parental cells (FIG. 2M). Collectively, the results support the conclusion that the oxidative efficiency of mitochondria was increased in the brain-seeking variants of MDA-MB-231 cells.

Identification of Four Candidate Metabolic Genes that could Account for the Brain Tropism of Human Breast Cancer Cells

Based on the working hypothesis of a metabolic preference of brain-seeking variants for metabolites present in the brain and on the above evidence of metabolic differences in the filiation, the next aim was to identify metabolic genes/proteins associated to the brain tropism of 231-BR and 231-BR-2 cells. Publicly available microarray database GEO #GSE66495 (reporting on the whole genome expression of not only of MDA-MB-231 and 231-BR cells, but also of MDA-MD-231-derived adrenal (ADMD-231), bone (BMD-231) and lung (LMD-231) metastatic variants) were analyzed, focusing on genes involved in glycolysis, the TCA cycle and OXPHOS.

Using the two-step methodology described in the materials and methods, 22 metabolic genes differentially expressed in at least one metastatic variant compared to parental MDA-MB-231 cells were identified, among which 6 were further differentially expressed in 231-BR cells compared to any other metastatic variant (Table 1). Significantly upregulated genes were ALDH9A1 (Genbank ID 223, on chromosome 1) encoding aldehyde dehydrogenase 9 family member A1, FH (Genbank ID 2271, on chromosome 1) encoding fumarate hydratase, and COX7B (Genbank ID 1349, on chromosome X), encoding cytochrome c oxidase subunit 7B. Significantly downregulated genes were ALDHIA3 (Genbank ID 220, on chromosome 15) encoding aldehyde dehydrogenase 1 family member A3, NDUFB8 (Genbank ID 4714, on chromosome 10) encoding NADH: ubiquinone oxidoreductase subunit B8, and PGM5 (Genbank ID 5239, on chromosome 9) encoding phosphoglucomutase 5.

TABLE 1
Metabolic genes differentially expressed in 231-BR versus other tissue-
specific variants of parental MDA-MB-231 human breast cancer cells

| | | ADMD-231 | | | | |
|---|---|---|---|---|---|---|
| | 231-BR | (adrenal | BMD-231 | LMD-231 | | |
| | (brain- | glands- | (bone- | (lung- | | |
| Gene ID | seeking) | seeking) | seeking) | seeking) | p⁴ | p⁵ |
| HK2¹ | 1.62^{2,3} | 1.83 | 1.66 | 1.70 | >0.05 | >0.05 |
| ALDH1A3 | 1.18 | 1.53 | 1.43 | 1.48 | <0.05 | >0.05 |
| ALDH9A1 | 1.55 | 1.04 | 1.07 | 1.09 | >0.05 | <0.05 |
| ALDH3A1 | 1.18 | 1.03 | 1.21 | 1.16 | >0.05 | >0.05 |
| PCK2 | 1.08 | −1.43 | −1.21 | −1.33 | <0.05 | <0.05 |
| IDH1 | −1.18 | −1.31 | −1.13 | −1.14 | >0.05 | >0.05 |
| FH | 1.54 | 1.08 | 1.04 | 1.11 | >0.05 | <0.05 |
| MDH2 | −1.10 | −1.19 | −1.17 | −1.27 | >0.05 | >0.05 |
| ATP5I | 1.25 | 1.36 | 1.33 | 1.33 | >0.05 | >0.05 |
| ATP5G2 | −1.06 | −1.21 | −1.07 | −1.11 | >0.05 | >0.05 |
| ATP6V0E2 | 1.62 | 1.43 | 1.41 | 1.34 | >0.05 | >0.05 |
| ATP6V1D | 1.69 | 2.45 | 1.83 | 1.96 | >0.05 | >0.05 |
| ATP6V0D1 | −1.02 | −1.23 | −1.14 | −1.11 | >0.05 | >0.05 |
| ATP6V0D2 | 1.21 | 1.08 | 1.03 | 1.20 | >0.05 | >0.05 |
| ATP6V1B1 | 1.25 | −1.09 | −1.07 | 1.03 | <0.05 | <0.05 |
| COX17 | −1.47 | −1.46 | −1.50 | −1.52 | >0.05 | >0.05 |
| COX7B | 2.02 | 1.06 | −1.06 | −1.20 | <0.05 | <0.05 |
| NDUFB7 | 1.45 | 1.54 | 1.27 | 1.21 | >0.05 | >0.05 |
| NDUFB8 | −1.43 | 1.46 | 1.45 | 1.54 | <0.05 | <0.05 |
| NDUFV3 | 1.10 | 1.51 | 1.42 | 1.37 | >0.05 | >0.05 |
| UQCRB | 1.31 | 1.05 | 1.27 | 1.30 | >0.05 | >0.05 |
| PGM5 | −1.11 | 6.42 | 4.32 | 4.51 | <0.05 | <0.05 |

¹From the GEO #GSE66495 microarray database (n = 3 per cell line), including only on genes involved in glycolysis, the TCA cycle and oxidative phosphorylation.
²Numbers report on fold changes compared to parental MDA-MB-231 cells used as control.
³Bold numbers are fold-changes with p < 0.05 compared to parental MDA-MB-231 cells using one-way ANOVA.
⁴p value for 231-BR compared to all other tissue-seeking variants using one-way ANOVA.
⁵p value for 231-BR compared to all other tissue-seeking variants and parental cells using one-way ANOVA. Genes of interest (p < 0.05) are highlighted in grey.

Among the 6 genes, ALDHIA3 and PGM5 expression was not significantly different (p>0.5) between 231-BR and parental MDA-MB-231 cells (Table 1). They were therefore excluded from further analysis.

### Cytochrome c Oxidase Subunit 7b in Mitochondrial Complex IV is a Candidate Protein Supporting the Brain Tropism of Human Breast Cancer Cells

The investigation was pursued with a short list of 4 genes: ALDH9A1, FH, NDUFB8, and COX7B. To avoid idiosyncrasies that would have been associated to 231-BR cells, changes in gene expression were independently tested using RT-qPCR in both 231-BR and 231-BR-2 brain-seeking variants. It was further verified that changes in protein matched changes in mRNA expression.

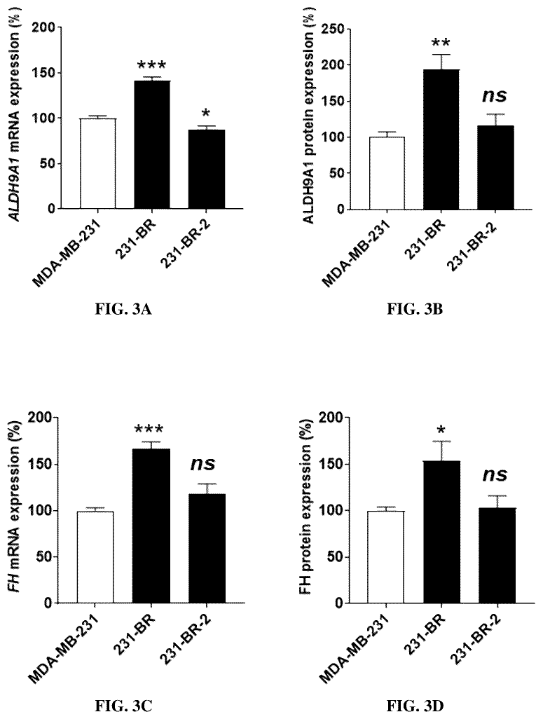

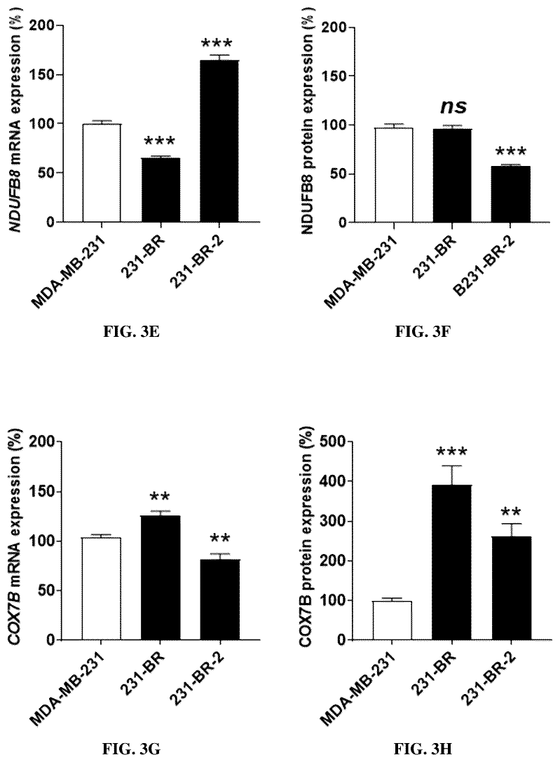

ALDH9A1 encodes a cytosolic aldehyde hydrogenase that catalyzes the oxidation of γ-aminobutyraldehyde and aminoaldehydes derived from polyamines. It is involved in carnitine biosynthesis, which facilitates the transport of fatty acids across the inner mitochondrial membrane for β-oxidation and, potentially, in a marginal pathway for the biosynthesis of neurotransmitter γ-aminobutyric acid (GABA). Compared to parental MDA-MB-231 cells, ALDH9A1 mRNA expression was significantly increased in 231-BR, but it was slightly decreased in 231-BR-2 cells (FIG. 3A). The corresponding protein was overexpressed in 231-BR, but not in 231-BR-2 cells (FIG. 3B), which disqualified it as a shared metabolic sensor for brain tropism in the model cell lines used herein.

FH encodes fumarate hydratase, the 7^{th} enzyme of the TCA cycle that catalyzes the hydration of fumarate to L-malate. When mutated/inactivated, FH can cause various diseases, including hereditary and sporadic forms of cancer. In the context of brain-specific breast cancer metastasis, FH mRNA and protein expression was increased in 231-BR but not in 231-BR-2 cells (FIG. 3C-3D), thus disqualifying this enzyme as a shared metabolic sensor for brain tropism.

NDUFB8 encodes an accessory subunit of NADH ubiquinone oxidoreductase, a large protein complex known as ETC Complex I at the inner mitochondrial membrane. The subunit is bound to NADH dehydrogenase 5 (ND5) in the proton-pumping module of Complex I. Similar to microarray data analysis, RT-qPCR showed significantly reduced NDUFB8 mRNA expression in 231-BR compared to MDA-MB-231 cells (FIG. 3E). However, it was significantly increased in 231-BR-2 cells, and changes in protein expression did not match changes in mRNA expression (FIG. 3E-F). Overall, this disqualified NDUFB8 as a metabolic sensor for brain-selective metastasis.

COX7B encodes subunit 7B of cytochrome c oxidase (COX), a large protein complex known as ETC Complex IV that catalyzes the transfer of electrons from reduced cytochrome c to molecular oxygen at the inner mitochondrial membrane. COX7B is a short 80 amino acid protein that stabilizes the complex and modulates COX activity. COX7B mRNA expression was increased in 231-BR but decreased in 231-BR-2 cells (FIG. 3G). However, the expression of the corresponding protein was increased in both variants (FIG. 3H). Considering that among the 4 candidates proteins, only COX7B protein expression showed a similar change in both brain-seeking variants, COX7B was retained for further investigation.

### COX7B Expression Drives Human Breast Cancer Cell Migration Towards Astrocytes

Transwell migration assays were used to establish a causal link between COX7B expression and MDA-MB-231 brain chemoattraction, mimicked by cell migration towards immortalized human and mouse astrocytes. Chemoattraction at other important metastatic sites was mimicked by immortalized human hepatocytes (T0063) and human bronchial epithelial cells (T0763) All four cell lines were nonmalignant.

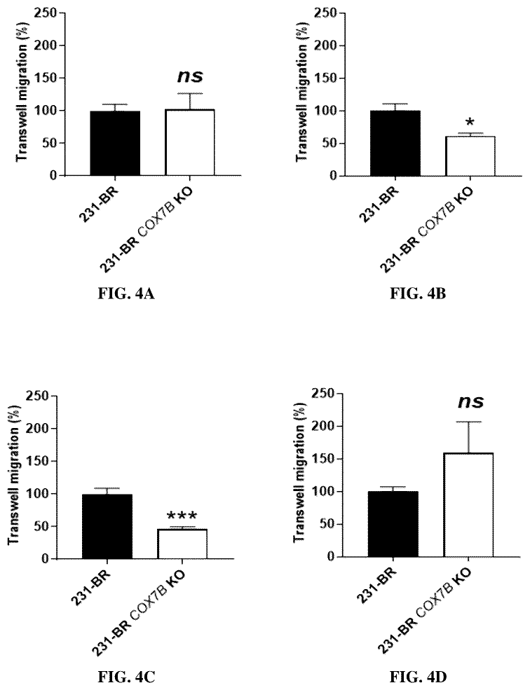

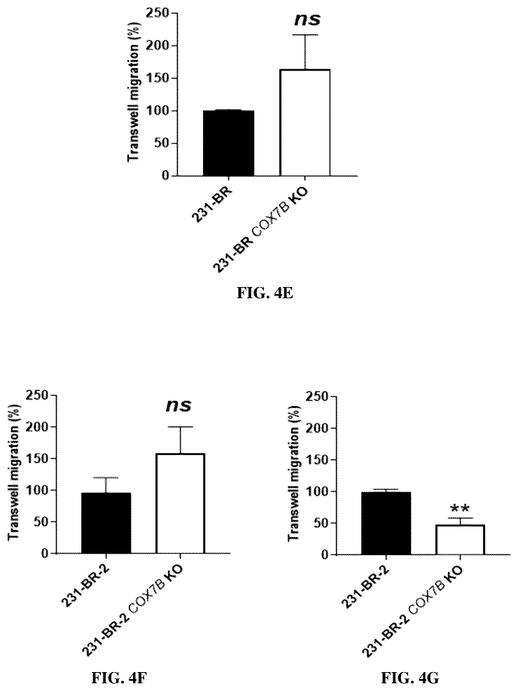

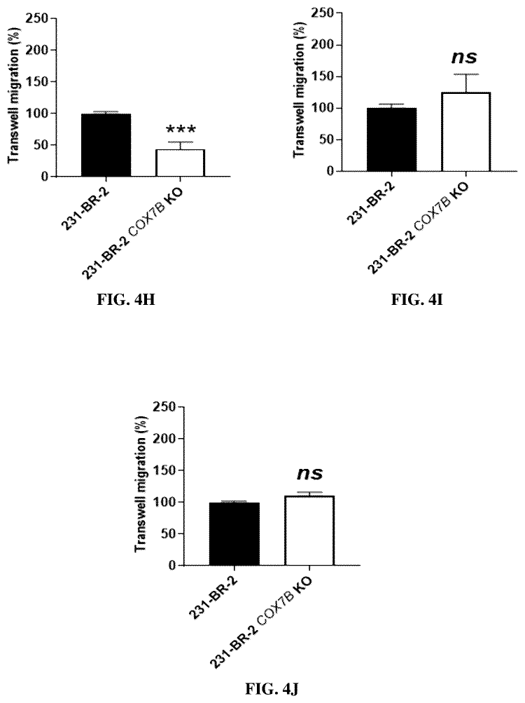

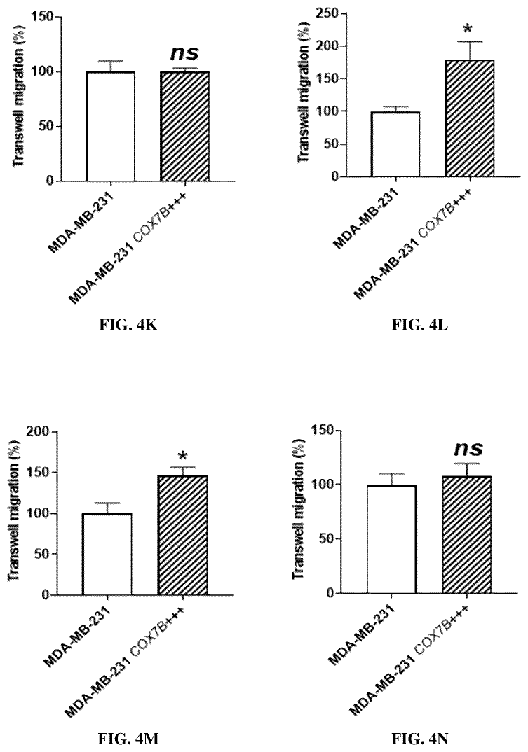

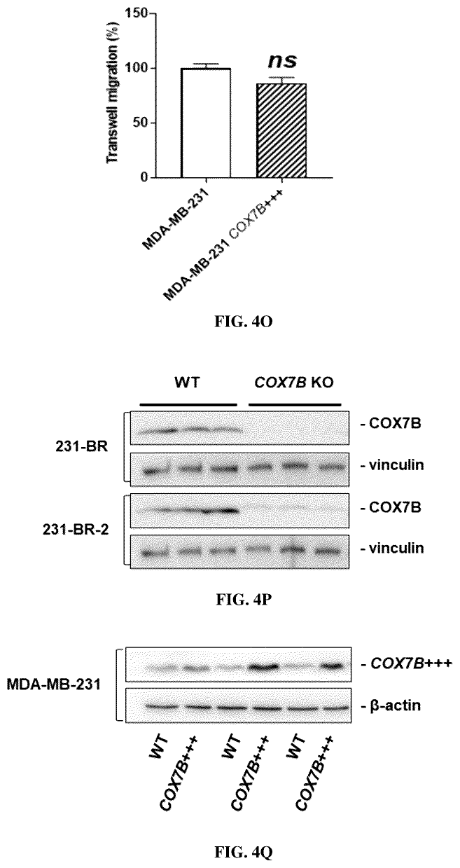

As expected, COX7B protein silencing in brain-seeking variant cells using a CRISPR-Cas9 strategy (FIG. 4P) significantly decreased 231-BR cell migration towards human and mouse astrocytes, but not towards human hepatocytes and human bronchial cells (FIG. 4A-4E). The general migration phenotype towards serum (1% FBS), used as a control, was unaffected. Similarly, COX7B silencing significantly reduced 231-BR-2 migration towards human and mouse astrocytes, but not towards human hepatocytes, human bronchial cells or serum (FIG. 4F-4J). Conversely, experimental COX7B protein overexpression in parental MDA-MB-231 cells (FIG. 4Q) increased cell migration towards human and mouse astrocytes, while migration towards human hepatocytes, human bronchial cells or serum was not changed (FIG. 4K-4O).

Together, these in vitro results supported a cause-effect relationship between COX7B protein expression and the brain tropism of human metastatic breast cancer cells. In particular, COX7B protein overexpression was sufficient to trigger a selective brain tropism of otherwise pan-metastatic, wild-type MDA-MB-231 cells.

### COX7B Expression Promotes the Oxidative Phenotype of Human Metastatic Breast Cancer Cells

The data displayed in FIG. 2A-2M showed that increased OXPHOS is a major metabolic characteristic of 231-BR and 231-BR-2 brain-seeking variants compared to parental cells. Because COX7B resides in the ETC, it was reasoned that its expression might modulate the OCR of the cells, which was measured using Seahorse oximetry.

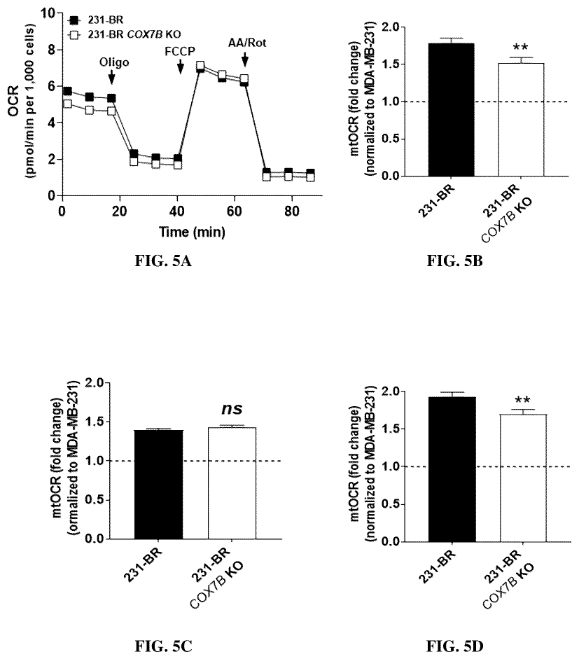

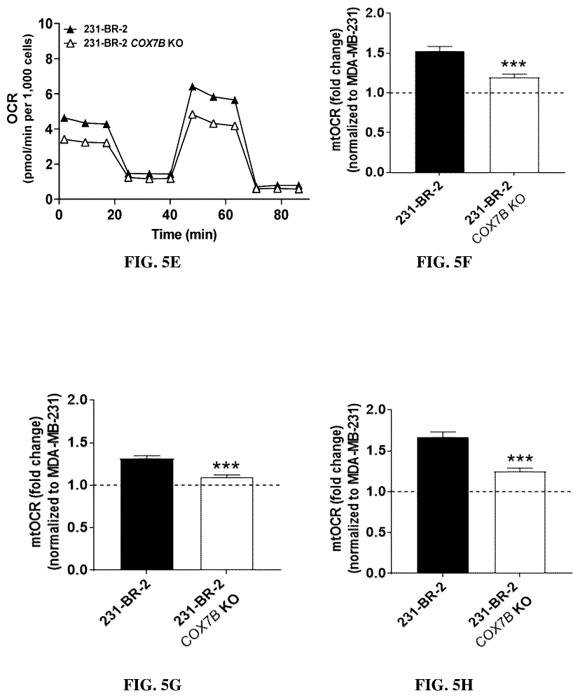

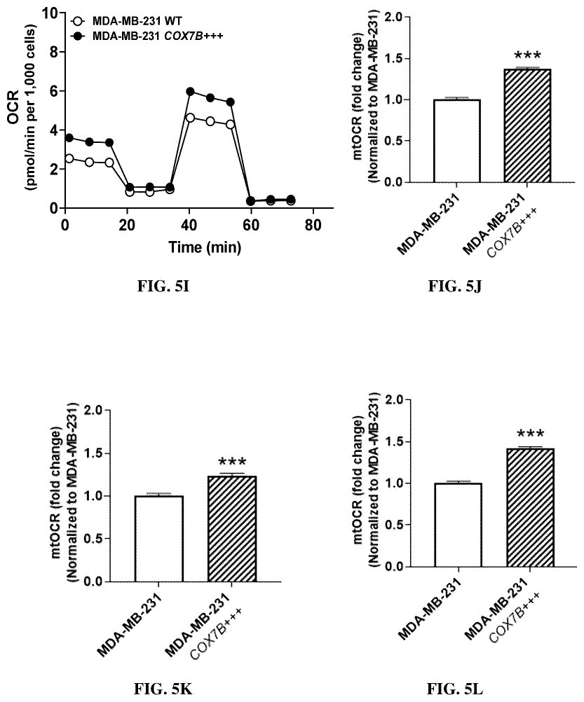

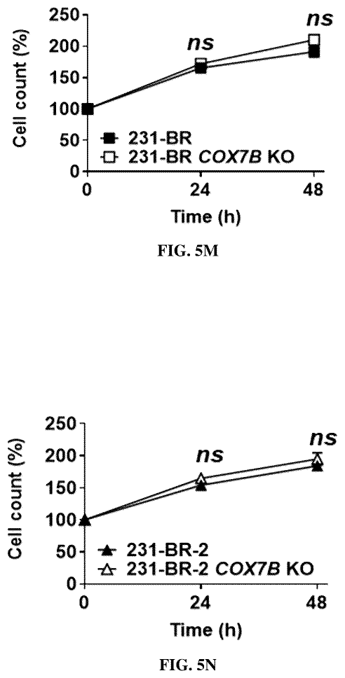

COX7B silencing reduced 231-BR basal OCR and OCR associated to ATP production, but maximal OCR, reflecting the respiration spare capacity, was unchanged (FIG. 5A-5D). Comparatively, COX7B silencing reduced all basal OCR, maximal OCR and OCR associated to ATP production in 231-BR-2 cells (FIG. 5E-5H), demonstrating that loss of COX7B represses OXPHOS in brain-seeking variants of metastatic breast cancer. Of note, COX7B silencing did not decrease cell numbers (FIG. 5M-5N), suggesting the existence of rescue metabolic pathways preventing cell death. Additionally, COX7B overexpression in wild-type MDA-MB-231 cells induced the opposite effect, i.e., an oxidative switch characterized by a raise in all basal OCR, maximal OCR and OCR associated to ATP production (FIG. 51-5L).

Together, these experiments demonstrated that COX7B is an OXPHOS inducer. They further established a positive correlation between the oxidative activities of human metastatic breast cancer cells and their preferential migration towards astrocytes.

### COX7B Expression is Responsible for the Brain Tropism of Metastatic Human Breast Cancer Cells in Mice

To experimentally establish a cause-effect relationship between COX7B expression and breast cancer brain metastasis, a series of in vivo experiments were ran in nude mice, as depicted in FIG. 1A. Briefly, because the present investigation interrogated metastatic tropism linked to metastatic take (a late metastatic event) but not metastatic cell dissemination from a primary tumor (an early metastatic event), breast cancer cells were injected in the left cardiac ventricle, which in the MDA-MB-231 generates systemic metastatic lesions to the bones, brain, ovary, and adrenal glands in MDA-MB-231 cells. The constitutive and concurrent expression of luciferase and GFP by the model cell lines used herein allowed to confirm bioluminescence data with immunohistochemistry.

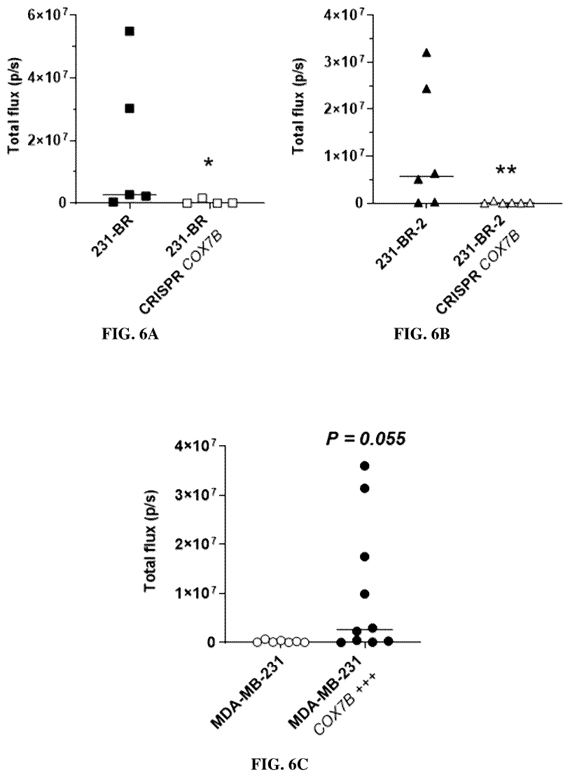

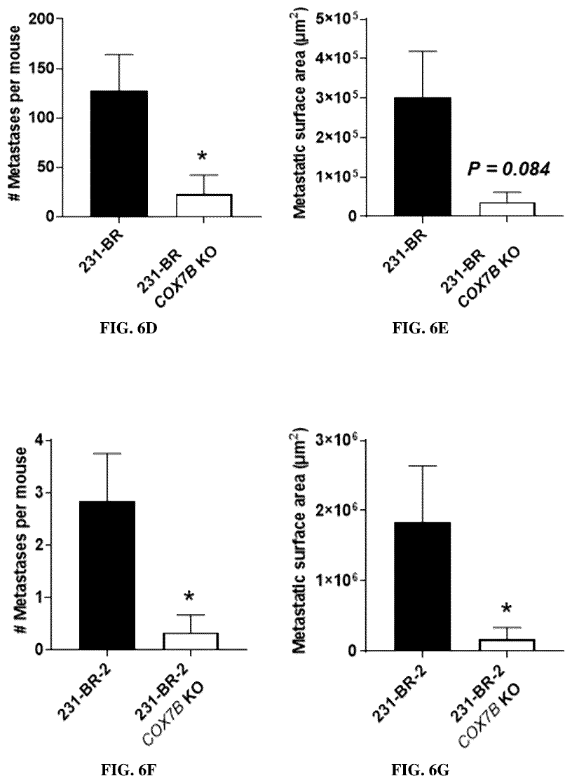

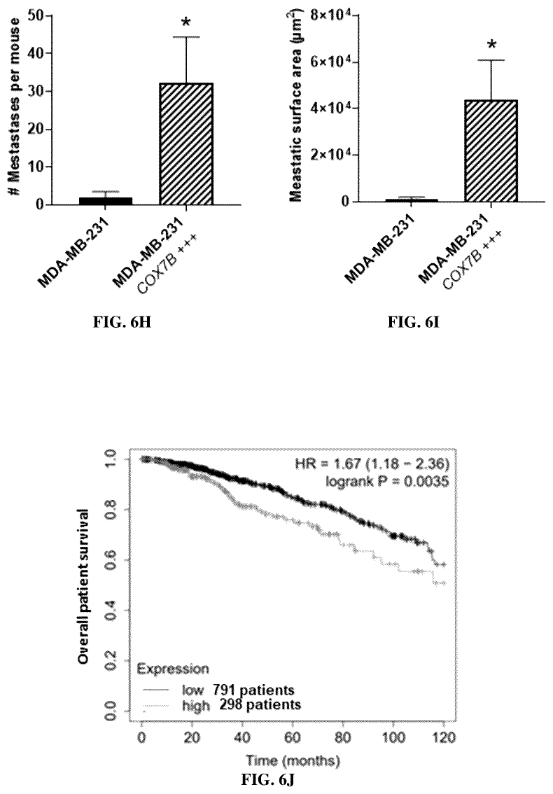

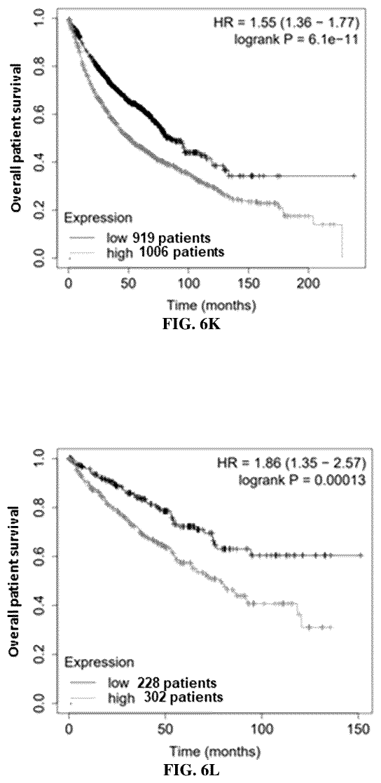

Following the protocol depicted in FIG. 1A, COX7B silencing with a CRISPR-cas9 strategy in 231-BR and 231-BR-2 brain-seeking variants resulted in an almost total loss of brain tropism following intracardiac injection (FIG. 6A-6C). This was evidenced using ex vivo luciferase bioluminescence imaging on brains isolated at the time of mouse sacrifice. Conversely, parental MDA-MB-231 cells, which did not generate detectable brain metastasis in 4 weeks, had a strong increase in brain metastases occurrence upon COX7b overexpression. For validation, brains were collected at the end of the experiments, sliced, stained with an antibody against GFP, and counterstained with hematoxylin and eosin. FIG. 6D-6I shows the number of metastases per mouse and the metastatic surfaces. Analyses revealed a strong decrease in the number of metastases per mouse and in the metastasis-positive tumor area per slice in mice injected with 231-BR and 231-BR-2 cells lacking COX7B compared to wild-type 231-BR and 231-BR-2 cells (FIG. 6D-6G). The opposite effects were seen in mice that received parental MDA-MB-231 cells overexpressing COX7B compared to wild-type MDA-MB-231 cells (FIG. 6H-6I). Collectively, these in vivo data established a cause-effect relationship between COX7B expression and the brain tropism of human TNBC in mice.

Publicly available gene chip and RNA-seq mRNA expression databases reporting on clinical human breast and lung cancers as well as on renal clear cell carcinoma, all subtypes included, were analyzed. High COX7B expression was identified as an independent poor prognosis factor for overall patient survival in all three cancer types (FIG. 6J-6L).

The findings described herein have an important implication in the field of rejuvenating brain therapy. Overexpressing COX7B in neuronal or mesenchymal stem cells, thus increasing their tropism to the brain following systemic injection, promotes the regeneration of neurons, oligodendrocytes, astrocytes and/or microglia.

### Example 2

Example 1 shows that high COX7B expression is responsible for brain-specific metastasis of human breast cancer cells MDA-MB-231, therefore it was reasoned that an experimentally induced overexpression of COX7B could serve to target cells to the brain in the context of cell therapy in neurodegenerative diseases, such as Alzheimer's disease, Parkinson's disease, Huntington's disease, amyotrophic lateral sclerosis and multiple sclerosis, and brain trauma, including ischemic stroke and accidental and surgical traumas, and lymphomas of the central nervous system. Cell therapy is indeed a promising strategy that can be used for neuroprotection (i.e., to delay the progression of neurodegenerative diseases) and/or brain repair (see, e.g., Steffanoni, S., et al., Diagnosis and Treatment Using Autologous Stem-Cell Transplantation in Primary Central Nervous System Lymphoma: A Systematic Review. Cancers (Basel), 2023. 15 (2)).

A main research area for brain repair and regeneration therapy is the use of stem cells, among which mesenchymal stem cells and neural stem cells are multipotent cells capable of self-renewal and able to differentiate into various central nervous system neuronal and glial cell types. Human mesenchymal stem cells can be isolated from various sources of the body, such as abdominal fat, bone marrow, and umbilical cord blood. Conversely, human neural stem cells originate from direct isolation from brain tissue and in vitro expansion, from the differentiation of pluripotent stem cells (such as human embryonic stem cells [hESCs] and induced pluripotent stem cells [iPSCs]) or from the transdifferentiation of somatic cells in culture. For brain therapy, these cells are typically transplanted intrathecally at the precise location of brain lesions.

While mesenchymal stem cells are used to suppress inflammation and to take advantage of their secretome that can provide neuroprotection and promote regeneration, neural stem cells produce neurotrophic factors, decrease neuroinflammation, enhance neuronal plasticity through integrating into host neuronal circuits, and replace damaged cells (Uchida, N., et al., Direct isolation of human central nervous system stem cells. Proc Natl Acad Sci USA, 2000. 97 (26): p. 14720-5).

Injecting stem cells in the brain faces two major issues. The first issue is the transplantation procedure itself, which raises safety, ethical and efficacy issues. It is invasive, should ideally be repetitive (i.e., chronic treatment involving several injections at different time points), and spatial administration depends on the capacity to detect brain lesions/degenerative areas. The second issue is the limited capacity of transplanted cells to migrate from the injection site to the widespread area of the affected sites, which is crucial for a therapeutic effect in the central nervous system (see, e.g., De Gioia, R., et al., Neural Stem Cell Transplantation for Neurodegenerative Diseases. Int J Mol Sci, 2020. 21 (9)). Typically, injected cells agglomerate and form clusters at the site of injection in the brain parenchyma, and the grafting efficiency/survival rate of the injected cells is estimated to be less than 5%. Stem cells can also be delivered by lumbar puncture injection into the cerebrospinal fluid. Currently, this procedure is more efficient than intravenous administration, notably because more cells are found in the injured site.

For stem cell therapy, optimizing the delivery of stem cells to the brain is thus an important task in translational research. Here, using ReNcell VM human neural progenitor cells as an example, it is demonstrated that overexpressing COX7B in stem cells promotes their homing to the brain parenchyma after systemic delivery in the blood stream.

### Materials and Methods

### Cells and Cell Culture

ReNcell VM human neural progenitor cells (Sigma-Aldrich, catalog #SCC008) were routinely grown in flasks coated with 1% of Matrigel (Corning, #356231) in proliferation medium DMEM F12 containing L-glutamine (Gibco, #11320-074), supplemented with 20 ng/ml hEGF (Sigma-Aldrich, #E9644-. 2 MG), 20 ng/ml hFGF (Sigma-Aldrich, #GF003AF-100UG), 2 μg/mL heparin (StemCell, #07980), 2% B27 supplement (ThermoFisher, #17504044) and 1% antibiotic-antimycotic (ThermoFisher, #15240062), in a humidified atmosphere with 5% CO₂ at 37° C.

### Genetic Manipulations

Expression vector pCMV3 encoding the human COX7B ORF cDNA sequence and possessing an hygromycin selection cassette (Sinobio, #HG20762-UT) was used to overexpress COX7B in ReNcell VM cells. Briefly, cells at 30-60% confluence in a 10 cm-dish were transfected with 10 μg of the plasmid using the Lipofectamine Stem Transfection Reagent (ThermoFisher, #STEM00001). They were allowed to recover for 24 h after transfection. Cells were then selected with 10 μg/mL hygromycin (InvivoGen, #ant-hg-1) for 14 days. Medium was renewed every 3-4 days. Colonies (clones) were then individually picked, expanded, and tested by western blotting for the expression of COX7B protein.

For constitutive luciferase and GFP expression, cells were infected with lentiviruses carrying the luciferase and GFP sequences along with a neomycin resistance gene (Amsbio, #LVP403). Briefly, 70-80% confluent cells in a 96-wells plate were transduced with 20 viral particles. The next day, the cells were selected by incubation with 400 μg/mL neomycin (InvivoGen, #ant-gn-1) during 10 days. After treatment, the cells were FACS-sorted for GFP expression on a Becton Dickinson FACSAria III system (Erembodegem, Belgium).

### Western Blotting

Western blotting was performed as previously described in Van Hee, V. F., et al (Van Hee, V. F., et al., Lactate does not activate NF-kappaB in oxidative tumor cells. Front Pharmacol, 2015. 6: p. 228), incorporated herein by reference. Membranes were incubated overnight with primary rabbit antibodies against COX7b (Abcam, #ab137094) or mouse antibodies against β-actin (Sigma-Aldrich, #A5441). Secondary antibodies were Peroxidase AffiniPure goat anti-Rabbit IgG (Jackson Immunoresearch, #111-035-003) and Peroxidase AffiniPure goat anti-mouse IgG (Jackson Immunoresearch, #115-035-003). Staining was revealed with an Amersham Imager 600 (Diegem, Belgium). All data were normalized to β-actin expression.

### In Vivo Assays

All in vivo experiments were conducted under approval of the Université catholique de Louvain (UCLouvain) authorities (Comité d'Ethique Facultaire pour l'Expérimentation Animale) according to national animal care regulations. Specific approval ID for this study was 2020/UCL/MD/033.

Brain damage was induced by LPS injections. Intraperitoneal injections of 1 μg/gram body weight (gbw) of LPS were performed for two consecutive days following the protocol of Bodea, L. G., et al., Neurodegeneration by activation of the microglial complement-phagosome pathway. J Neurosci, 2014. 34 (25): p. 8546-56, incorporated herein by reference.

Stem cells overexpressing COX7B or not were delivered in the blood stream of 18 weeks old female NMRI nude mice as described in Example 1 for the delivery of MDA-MB-231 cancer cells. All mice were sacrificed 3 days later by cervical dislocation under terminal anesthesia. Their brains were harvested, cut along the separation between right and left hemispheres, and embedded in paraffin.

### Immunohistochemistry

Collected brains were processed for immunohistochemistry. Sections (5 μm-thick) were performed from the center of each hemisphere to produce 10 slides for each sample, and fixed in 4% paraformaldehyde (PFA). Brain sections were then immunostained with an anti-GFP antibody (NovusBio, #NB-600 308) and with a secondary Envision anti-rabbit antibody coupled to HRP (Agilent, #K4003). All slices were counterstained with hematoxylin and eosin. Slides were scanned at 20× magnification with a bright field Panoramic Scan II microscope (3DHISTECH Kft. Budapest, Hungary). Images were analyzed with SlideViewer (3DHISTECH Kft. Budapest, Hungary) and the QuPath software version 0.1.2 (Belfast, UK).

### Results

### Overexpression of COX7B in Human Neural Progenitor Cells

It was thus tested whether COX7B overexpression in stem cells could promote their homing to a damaged brain after the systemic delivery of the cells in the blood stream. As cellular model, ReNcell VM human neural progenitor cell line (ReN) was used, which is an immortalized cell line that can be maintained for more than 45 passages in culture and can differentiate into neurons and glial cells with simple growth-factor deprivation. This cell line has already been used by others to study brain engraftment after intrathecal injection in rats (see, e.g., Hovakimyan, M., et al., Survival of transplanted human neural stem cell line (ReNcell VM) into the rat brain with and without immunosuppression. Ann Anat, 2012. 194 (5): p. 429-35.)

To study the homing of the stem cells to damaged brain, ReNcell VM cells were first genetically modified to express high levels of the COX7B protein. To induce COX7B overexpression, the pCMV3 plasmid carrying the human untagged COX7B open reading frame cDNA sequence as well as an hygromycin resistance sequence were used. Selection after transfection allowed to retrieve 14 ReNcell VM cell clones that expressed higher levels of the COX7B protein compared to the wild-type parental cells (FIG. 8). To preserve the diversity of the initial cell population, these 14 clones were pooled together to create the ReNcell VM COX7B OE stem cell line.

Demonstration that the Homing of Human Neural Progenitor Cells Overexpressing COX7B in a Damaged Brain is Increased Compared to Human Neural Progenitor Cells that do not Overexpress COX7B

For in vivo cell tracking, ReNcell VM and ReNcell VM COX7B OE cells were modified to constitutively express luciferase and the green fluorescent protein (GFP). A lentivirus carrying the luciferase and GFP sequences along with a neomycin resistance cassette was used. After selection, ReNcell VM cells with constitutive luciferase and constitutive GFP expression were obtained on the one hand, and ReNcell VM COX7B OE cells with constitutive luciferase and constitutive GFP expression were obtained on the other hand. The two cell lines were used to compare their homing in damaged mouse brain. NMRI nude mice treated with LPS (1 μg/gbw) were used, because this strain is compatible with the engraftment of human cells and because LPS was previously shown to induce neuroinflammation resulting in brain damage, which is now widely used as a model of brain damage and neurodegeneration (Trepanier, M. O., et al., Increased brain docosahexaenoic acid has no effect on the resolution of neuroinflammation following intracerebroventricular lipopolysaccharide injection. Neurochem Int, 2018. 118: p. 115-126; Bodea, L. G., et al., Neurodegeneration by activation of the microglial complement-phagosome pathway. J Neurosci, 2014. 34 (25): p. 8546-56).

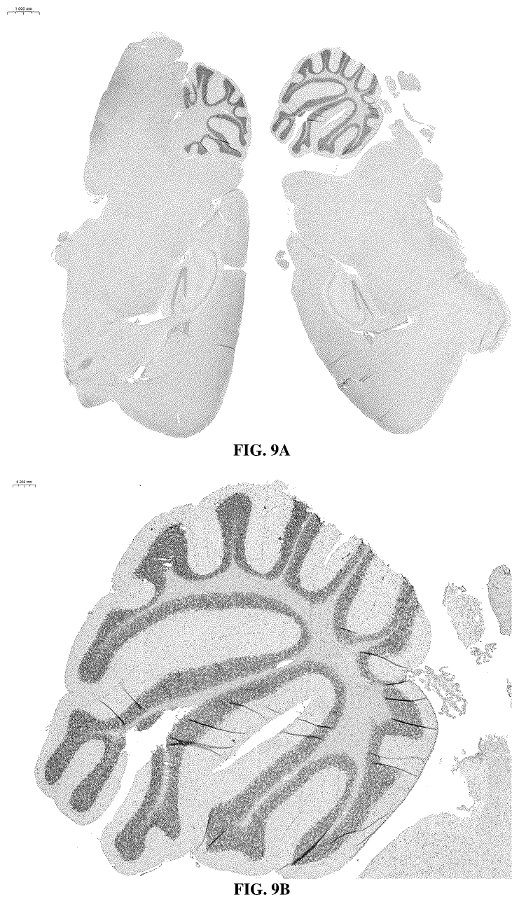

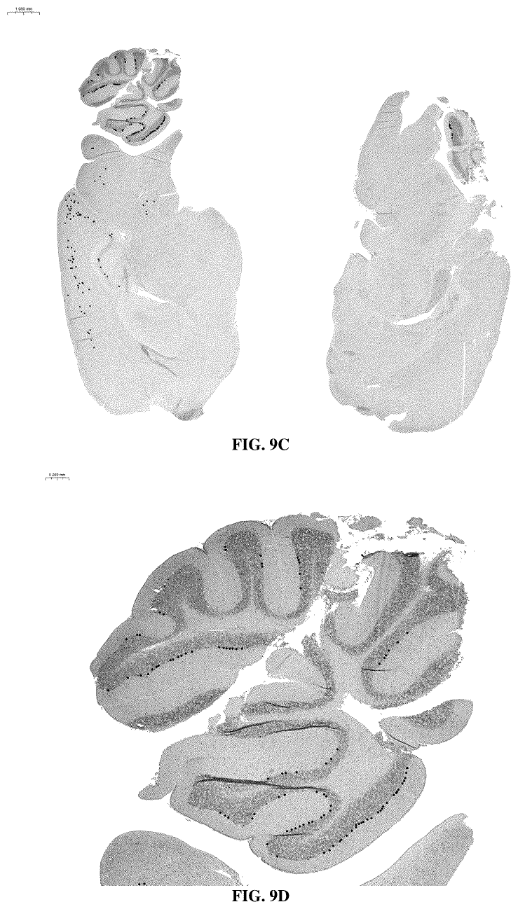

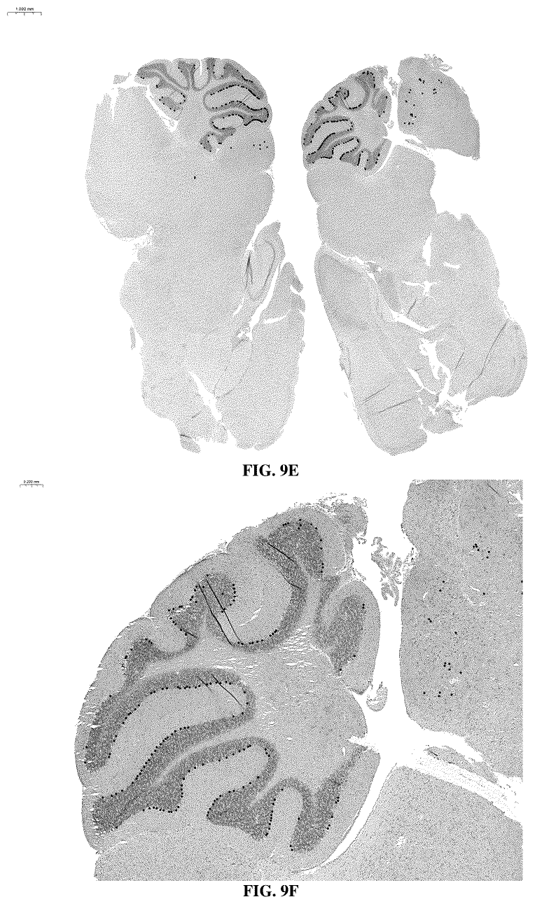

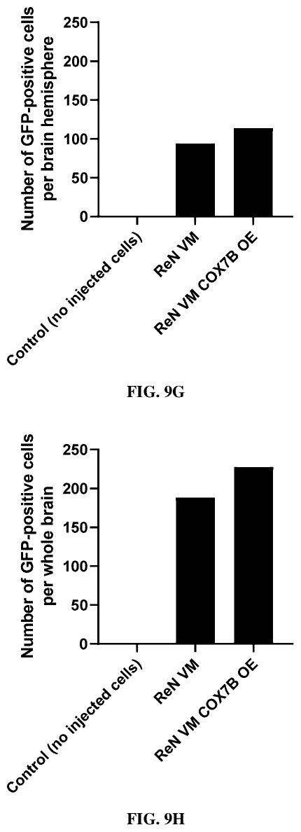

Three weeks after LPS treatment, mice received a bolus injection of either 100.000 of ReNcell VM cells or 100.000 ReNcell VM COX7B OE cells in the systemic blood circulation. All mice were sacrificed by cervical dislocation under terminal anesthesia 3 days after stem cell injection, and brains were collected and embedded in paraffin. For each mouse, a series of sections of the two brain hemispheres were immunostained for GFP expression, allowing to detect the injected human stem cells with full certitude. The results presented in FIG. 9A-9H unequivocally show that COX7B overexpression improved the homing of human neural stem cells to the damaged brain of immunocompromised mice. Within the brain parenchyma, ReNcell VM COX7B OE cells were widely distributed in the cortex along areas that also stained positively for brain cell bodies, which are known to host a dense brain vasculature.

### CONCLUSIONS

The experimental data disclosed hereinabove support that the intervention consisting in overexpressing COX7B in human stem cells improves the homing of these cells to a damaged brain. While mice were used for the in vivo demonstration in Example 2, Example 1 demonstrates that COX7B overexpression allows the tropism to target cells not only to mouse astrocytes but also to human astrocytes.

Therefore, all experimental evidence provided herein demonstrate that the strategy consisting in overexpressing COX7B in stem cells before their injection in the blood stream improves their engraftment into the parenchyma of a damaged brain. For preventive/regenerative cell therapy of the brain, the present invention allows to exploit a less invasive, safer and potentially more efficient (in term of spatial distribution) administration route than stem cell transplantation in the brain or injection in brain fluids.

Of important note, it was globally demonstrated that engineering a human cancer cell line and a human stem cell line to increase COX7B expression improved their homing to the brain, which allows to generalize the finding by stating that overexpressing COX7B in human cells in general should increase their tropism to the brain after injection in the blood stream.

1-15. (canceled)

16. A method for treating and/or preventing a brain disease or disorder in a subject in need thereof comprising administering to the subject a population of cells overexpressing the cytochrome c oxidase subunit VIIb (COX7B) protein.

17. The method according to claim 16, wherein said cells are stem cells.

18. The method according to claim 16, wherein said cells are mesenchymal stem cells or neural stem cells.

19. The method according to claim 16, wherein said cells are mammalian cells.

20. The method according to claim 16, wherein said cells are human cells.

21. The method according to claim 16, wherein said cells originate from said subject.

22. The method according to claim 16, wherein said COX7B protein is overexpressed at a level so as to induce the brain tropism of said cells.

23. The method according to claim 16, wherein said cells stably overexpress COX7B protein.

24. The method according to claim 16, wherein said brain disease or disorder is selected from the group consisting of neurodegenerative diseases, neurological disorders, brain injuries and cerebrovascular diseases.

25. The method according to claim 16, wherein said brain disease or disorder is selected from the group consisting of Alzheimer's disease, Parkinson's disease, Huntington's disease, dementia, diffuse Lewy Body dementia, frontotemporal dementia (FTD), Louis body dementia, ataxia, motor neuron disease, amyotrophic lateral sclerosis (ALS), epilepsy and seizures, multiple system atrophy, multiple sclerosis, leukodystrophy, progressive supranuclear palsy, Olivopontocerebellar atrophy (OPCA), Shy-Drager syndrome, Striatonigral degeneration, cortico-basal ganglionic degeneration, Parkinson-ALS-dementia complex of Guam and Pick's disease, amyloidosis, Pick's disease, Lou Gehrig's disease, Creutzfeld-Jakob disease, mild cognitive impairment, syphilis, attention deficit hyperactivity disorder (ADHD), schizophrenia, depression, manic-depression, stress disorder, spinal cord injury, myelitis, Lugeric's disease, traumatic brain injury (TBI), concussion, vascular dementia, hypertension, toxic brain injury, hypoxia, carbon monoxide poisoning, encephalitis, stroke, brain tumors, brain abscess, and autism spectrum disorder.

26. The method according to claim 16, wherein said method comprises administering to the subject a therapeutic effective amount from about 1,000 to about 10,000,000 million of said cells per kg of body weight.

27. The method according to claim 16, wherein said population of cells is to be systemically administered to said subject.

28. The method according to claim 16, wherein said population of cells is to be intravenously injected to said subject.

29. The method according to claim 16, wherein said population of cells is to be administered in combination with another therapeutic agent.

30. A method for preventing and/or treating a brain disease or disorder in a subject in need thereof, comprising administering to the subject a pharmaceutical composition comprising a population of cells overexpressing COX7B protein, and a pharmaceutically acceptable vehicle.

31. A combination kit comprising (i) a population of cells overexpressing COX7B protein or a pharmaceutical composition comprising the same or means for overexpressing COX7B protein in cells and (ii) another therapeutic agent for use for the prevention and/or the treatment of a brain disease or disorder.

32. A method for inducing the brain tropism of cells, comprising a step of overexpressing COX7B protein in said cells.

33. The method according to claim 32, wherein said cells are stem cells.


SEQUENCE LISTING (16 sequences)

SEQ ID NO: 1 - protein, 80 aa, Homo sapiens
MFPLVKSALNRLQVRSIQQTMARQSHQKRTPDFHDKYGNAVLASGATFCIVTWTYVATQVGIEWNLSPVGRVTPKEWRNQ

SEQ ID NO: 2 - DNA, 20 bp, synthetic construct
  source 1..20: mol_type = other DNA
aaggagcagggtgctaaagt

SEQ ID NO: 3 - DNA, 25 bp, synthetic construct
  source 1..25: mol_type = other DNA
tcgtctctgcaattagttaatacac

SEQ ID NO: 4 - DNA, 22 bp, synthetic construct
  source 1..22: mol_type = other DNA
tgccaaccccagttattaaagc

SEQ ID NO: 5 - DNA, 21 bp, synthetic construct
  source 1..21: mol_type = other DNA
cttcagctacctcatctgctg

SEQ ID NO: 6 - DNA, 19 bp, synthetic construct
  source 1..19: mol_type = other DNA
cggatgatggcatggggta

SEQ ID NO: 7 - DNA, 18 bp, synthetic construct
  source 1..18: mol_type = other DNA
ggtgccagtgcatcggtt

SEQ ID NO: 8 - DNA, 20 bp, synthetic construct
  source 1..20: mol_type = other DNA
tacctgaagcgaattggcac

SEQ ID NO: 9 - DNA, 20 bp, synthetic construct
  source 1..20: mol_type = other DNA
gcttcgaacttggagacgat

SEQ ID NO: 10 - DNA, 16 bp, synthetic construct
  source 1..16: mol_type = other DNA
cccgcgagcacagagc

SEQ ID NO: 11 - DNA, 21 bp, synthetic construct
  source 1..21: mol_type = other DNA
tcatcatccatggtgagctgg

SEQ ID NO: 12 - DNA, 22 bp, synthetic construct
  source 1..22: mol_type = other DNA
gtacccacgtaaagacgttagg

SEQ ID NO: 13 - DNA, 20 bp, synthetic construct
  source 1..20: mol_type = other DNA
tactgctaaatccaccttcg

SEQ ID NO: 14 - DNA, 20 bp, synthetic construct
  source 1..20: mol_type = other DNA
cccatgaggtggcaagaaat

SEQ ID NO: 15 - DNA, 20 bp, synthetic construct
  source 1..20: mol_type = other DNA
agcgcactaaatcgtctcca

SEQ ID NO: 16 - DNA, 20 bp, synthetic construct
  source 1..20: mol_type = other DNA
gagttaccccaaaggaatgg